(19) United States
(12) Patent Application Publication
(10) Pub. No.: US 2025/0186555 A1
(43) Pub. Date: Jun. 12, 2025

(54) FORMULATIONS COMPRISING ACID-NEUTRALIZING POLYMER FOR ORAL ADMINISTRATION OF GLUCAGON-LIKE PEPTIDE-2

(71) Applicant: Entera Bio Ltd., Jerusalem (IL)

(72) Inventors: Gregory BURSHTEIN, Modiin (IL); Constantin ITIN, Mevaseret-Zion (IL); Hillel GALITZER, Yad Binyamin (IL); Phillip M. SCHWARTZ, Jerusalem (IL)

(73) Assignee: Entera Bio Ltd., Jerusalem (IL)

(21) Appl. No.: 18/841,378

(22) PCT Filed: Feb. 23, 2023
(86) PCT No.: PCT/IL2023/050195

§ 371 Date: Aug. 25, 2024

Related U.S. Application Data
(60) Provisional application No. 63/313,361, filed on Feb. 24, 2022.

(51) Int. Cl. A61K 38/26; A61K 9/00; A61K 9/20

(52) U.S. Cl. CPC A61K 38/26; A61K 9/0053; A61K 9/2013; A61K 9/2054; A61K 9/2059

(57) ABSTRACT

A pharmaceutical composition is described herein, which comprises a therapeutically active agent, an absorption enhancer, and a polymer comprising a plurality of alkaline groups. The therapeutically active agent is a glucagon-like peptide-2. A concentration of the polymer in the composition is at least 10 weight percent of the total weight of the composition. The absorption enhancer is preferably a substituted or non-substituted fatty acid or a salt thereof. Further described herein are methods of treating a condition treatable by the therapeutically active agent, comprising orally administering the pharmaceutical composition.

### RELATED APPLICATION/S

This application claims the benefit of priority under 35 USC § 119(e) of U.S. Provisional Patent Application No. 63/313,361 filed on Feb. 24, 2022, the contents of which are incorporated herein by reference in their entirety.

This application is also related to co-filed PCT International Patent Application having Attorney's Docket No. 95409, entitled “FORMULATIONS COMPRISING ACID-NEUTRALIZING POLYMER FOR ORAL ADMINISTRATION OF ACTIVE AGENTS”, which claims the benefit of priority under 35 USC § 119(e) of U.S. Provisional Patent Application No. 63/313,363 filed on Feb. 24, 2022, the contents of which are incorporated herein by reference in their entirety.

### FIELD AND BACKGROUND OF THE INVENTION

The present invention, in some embodiments thereof, relates to drug delivery, and more particularly, but not exclusively, to formulations and/or systems for oral administration of therapeutically active agents, such as, but not limited to, a glucagon-like peptide-2.

Human glucagon-like peptide-2 (GLP-2) is a 33-amino acid polypeptide produced by intestinal endocrine L cells and by central nervous system neurons. GLP-2 acts by binding to GLP-2 receptor; and enhances intestinal function, reduces bone breakdown and promotes neuroprotection. GLP-2 and other GLP-2 receptor agonists may be used for treating short bowel syndrome, Crohn's disease, osteoporosis, and conditions associated with a bone fracture or bone defect, and as adjuvant therapy during cancer therapy.

Teduglutide, which has been approved for treatment of short bowel syndrome, is a GLP-2 homolog in which an alanine is replaced by a glycine, increasing the half-life in humans from about 7 minutes to about two hours. Apraglutide and glepaglutide are GLP-2 homologs designed to have an even longer half-life than teduglutide [Hargrove et al., J Pharmacol Exp Ther 2020, 373:193-203; Naimi et al., EBioMedicine 2019, 46:444-451].

Oral administration of peptide and/or protein pharmaceuticals is problematic due to degradation of peptides and/or proteins in the digestive system and poor absorption of large molecules.

Qi & Ping [J Microencapsulation 2004, 21:37-45] describe administration of enteric microspheres containing insulin with SNAC (sodium 8-N-(2-hydroxybenzoyl)aminocaprylate). The enteric microspheres are for protecting the insulin from digestive enzymes of the stomach and small intestine, and the SNAC is for enhancing absorption.

U.S. Patent Application Publication No. 2011/0142800 describes compositions for oral administration of a protein, comprising a protein having a molecular weight of up to 100,000 Da, a protease inhibitor, and an absorption enhancer, such as SNAC, N-(10-[2-hydroxybenzoyl]amino)decanoic acid (SNAD), 8-[N-(2-hydroxy-4-methoxybenzoyl)amino]caprylic acid (4-MOAC), 8-[N-(2-hydroxy-5-chlorobenzoyl)amino]caprylic acid (5-CNAC) and 4-[(4-chloro-2-hydroxy-benzoyl)amino]butanoic acid (4-CNAB) and sodium salts thereof.

International Patent Application Publication No. WO 00/48589 describes solid oral dosage forms comprising a heparin drug in admixture with SNAC or SNAD for facilitating absorption and/or enhancing bioavailability of the heparin drug, wherein the heparin drug is reported to protect the SNAC or SNAD from precipitation during transit through acidic regions of the gastrointestinal (GI) tract.

International Patent Application Publication No. WO 2016/128974 describes a pharmaceutical composition for oral administration comprising a therapeutically active agent, SNAC and at least one antacid compound; as well as a pharmaceutical composition unit dosage form for oral administration which comprises a core comprising a therapeutically active agent and SNAC, and an external layer comprising at least one of an antacid compound and a protease inhibitor.

Buckley et al. [Sci Transl Med 2018, 10:eaar7047] reports that upon oral administration of a tablet comprising the peptide agent semaglutide and SNAC, absorption of the semaglutide takes place in the stomach and is confined to an area in close proximity to the tablet surface, and that the SNAC protects against enzymatic degradation via local buffering actions and only transiently enhances absorption.

Sodium carboxymethylcellulose (CMC) is prepared by modifying cellulose with chloroacetic acid, and may be crosslinked or non-crosslinked. In its crosslinked form, wherein crosslinking is effected by formation of ester bonds between carboxymethyl groups and cellulose, it is commonly referred to as croscarmellose sodium (CCS) or sodium croscarmellose, and is used as a superdisintegrant in pharmaceutical formulations.

Similarly, sodium starch glycolate (SSG) can be prepared by modifying starch with chloroacetic acid to form carboxymethyl groups. The sodium starch glycolate may be further crosslinked by phosphate groups (—O—P(═O)(—O⁻)—O—) between starch backbones, and used as a superdisintegrant.

Additional background art includes Qi et al. [Acta Pharm Sinica 2004, 39:844-848]; International Patent Application Publication Nos. WO 00/50386, WO 01/32130, WO 01/32596, WO 03/045306, WO 03/045331, WO 2006/076692, WO 2007/121471, WO 2010/020978, WO 2012/080471, WO 2016/128970, WO 2016/128971, WO 2016/128972, WO 2016/128973, WO 2016/128974 and WO 2018/033927; Japanese Patent Application Nos. 2005281231 and 2006111558; U.S. Pat. No. 8,110,547; and U.S. Patent Application Publication Nos. 2006/0234913, 2007/0087957, 2013/0224300 and 2020/0000883.

### SUMMARY OF THE INVENTION

According to an aspect of some embodiments of the invention, there is provided a pharmaceutical composition comprising a therapeutically active agent, an absorption enhancer, and a polymer comprising a plurality of alkaline groups. According to embodiments of the present invention, the therapeutically active agent is or comprises a glucagon-like peptide-2.

According to embodiments of the present invention, a concentration of the polymer in the composition is at least 10 weight percent of the total weight of the pharmaceutical composition.

According to embodiments of the present invention, the absorption enhancer is a substituted or non-substituted fatty acid or a salt thereof.

According to an aspect of some embodiments of the invention, there is provided a method of treating a condition treatable by a therapeutically active agent as described herein in a subject in need thereof, the method comprising orally administering to the subject a pharmaceutical composition according to any of the respective embodiments described herein.

According to some of any of the embodiments of the invention, the alkaline groups are carboxylate groups and/or amine groups.

According to some of any of the embodiments of the invention, at least a portion of the alkaline groups are carboxylate groups.

According to some of any of the embodiments of the invention relating to carboxylate groups, at least a portion of the carboxylate groups are in a form of a pharmaceutically acceptable salt.

According to some of any of the embodiments of the invention relating to carboxylate groups, at least a portion of the carboxylate groups are in a form of a sodium salt.

According to some of any of the embodiments of the invention, the absorption enhancer is selected from NAC (8-N-(2-hydroxybenzoyl)aminocaprylic acid), NAD (10-N-(2-hydroxybenzoyl)aminodecanoic acid), 5-CNAC (8-N-(5-chlorosalicyloyl)aminocaprylic acid), 4-MOAC (8-N-(2-hydroxy-4-methoxybenzoyl)aminocaprylic acid), 4-CNAB (4-N-(2-hydroxy-4-chlorobenzoyl)aminobutanoic acid) and salts thereof.

According to some of any of the embodiments of the invention, the absorption enhancer comprises NAC or a salt thereof.

According to some of any of the embodiments of the invention, a concentration of the absorption enhancer is at least 50 weight percent of the total weight of the pharmaceutical composition.

According to some of any of the embodiments of the invention, a concentration of the polymer is at least 20 weight percent of the total weight of the pharmaceutical composition.

According to some of any of the embodiments of the invention, a total concentration of the absorption enhancer and the polymer is at least 80 weight percent of the total weight of the pharmaceutical composition.

According to some of any of the embodiments of the invention, a concentration of the alkaline groups in the pharmaceutical composition is at least 0.1 millimoles per gram.

According to some of any of the embodiments of the invention, the polymer is a crosslinked polymer.

According to some of any of the embodiments of the invention, the polymer comprises a polysaccharide.

According to some of any of the embodiments of the invention relating to a polysaccharide, the polysaccharide is selected from a starch derivative and a cellulose derivative.

According to some of any of the embodiments of the invention, the polymer comprises carboxymethyl groups.

According to some of any of the embodiments of the invention, the polymer is characterized by a pKa in a range of from 1.2 to 7.5.

According to some of any of the embodiments of the invention, the polymer is sodium starch glycolate and/or croscarmellose sodium.

According to some of any of the embodiments of the invention, a Cmax and/or a bioavailability of the pharmaceutical composition upon oral administration is at least 50% greater than a Cmax and/or a bioavailability of a corresponding composition without the polymer (e.g., a composition comprising the same therapeutically active agent, the same absorption enhancer and optionally other ingredient(s), each in the same amount, but devoid of a polymer comprising a plurality of alkaline groups as described herein in any of the respective embodiments).

According to some of any of the embodiments of the invention, the therapeutically active agent is selected from teduglutide, apraglutide, dapiglutide and glepaglutide.

According to some of any of the embodiments of the invention, the composition is in a form of a unit dosage form.

According to some of any of the embodiments of the invention relating to a unit dosage form, an amount of the alkaline groups in the unit dosage form is at least 0.03 millimoles.

According to some of any of the embodiments of the invention relating to a unit dosage form, the unit dosage form comprises at least 50 mg of the absorption enhancer.

According to some of any of the embodiments of the invention relating to a unit dosage form, the unit dosage form comprises one or more tablet(s).

According to some of any of the embodiments of the invention, the composition is for use in the treatment of a condition treatable by the therapeutically active agent, the treatment comprising oral administration of the pharmaceutical composition.

According to some of any of the embodiments of the invention relating to treatment of a condition, the condition is selected from chemotherapy-induced adverse effects, cul-de-sac syndrome, digestion disorders, enteritis, inflammatory bowel disease, malabsorption syndromes, malnutrition, short-bowel syndrome, small intestine damage, celiac sprue, tropical sprue, hypogammaglobulinemic sprue, ulcers, and ulcerative colitis.

According to some of any of the embodiments of the invention relating to treatment of a condition, the condition is short-bowel syndrome.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents a graph showing the measured pH upon addition of SNAC to an HCl solution (pH 1.2), as a function of SNAC concentration.

FIGS. 2A-B present graphs showing the pH upon addition of sodium alginate, sodium carboxymethylcellulose (Na-CMC), sodium starch glycolate (SSG) or croscarmellose sodium (CCS) to an HCl solution (pH 1.2), as a function of concentration of the added polymer, measured using MP-103 pH-meter (MRC, Israel) equipped with ELC-10-00 electrode (MRC, Israel) (FIG. 2A) or with a thin electrode HI1083 (HANNA instruments Inc.) (FIG. 2B).

FIGS. 3A-D present photographs of tablets made of the polymers sodium starch glycolate (SSG; FIG. 3A), sodium croscarmellose (CCS; FIG. 3B), sodium carboxymethyl cellulose (CMC-Na; FIG. 3C) and sodium alginate (Alg-Na; FIG. 3D), following swelling in 0.01 M HCl (pH=2), at room temperature, along with pH values measured at four points for each tablet (white triangles).

FIGS. 4A-B present photographs of tablets made of the polymers sodium starch glycolate (SSG; FIG. 4A), and sodium carboxymethyl cellulose (CMC-Na; FIG. 4B), following swelling in porcine gastric juice, at room temperature, along with pH values measured at four points for each tablet (white triangles).

FIG. 5 is a graph showing the median teduglutide plasma concentration following oral administration of tablets comprising 0.7 mg teduglutide (2.34 mg/Kg), SNAC (15 mg) and SSG (5.33 MG) to male Sprague-Dawley rats weighing about 299 grams, n=7.

### DESCRIPTION OF SPECIFIC EMBODIMENTS OF THE INVENTION

The present invention, in some embodiments thereof, relates to drug delivery, and more particularly, but not exclusively, to formulations and/or systems for oral administration of therapeutically active agents, such as, but not limited to, a glucagon-like peptide-2.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

While investigating the enhancement of absorption of therapeutically active agents by an exemplary absorption enhancer such as SNAC (sodium 8-N-(2-hydroxybenzoyl)aminocaprylate), the present inventors have uncovered that the ability of such absorption enhancers to enhance absorption (e.g., as determined by bioavailability and/or Cmax) of therapeutically active agents is adversely affected by the acidic environment of the stomach.

The inventors have conceived using basic (alkaline) compounds to enhance the performance of absorption enhancers upon oral administration by neutralizing stomach acid, and uncovered that polymers comprising alkaline groups such as carboxylate are particularly effective at protecting the absorption enhancer from stomach acid, as well as enhancing bioavailability of a therapeutically active agent in compositions comprising the absorption enhancer and the therapeutically active agent.

While reducing the present invention to practice, the inventors have shown that exemplary polymers comprising carboxylate groups produce a local increase in pH in simulated gastric fluid (which increase is capable of reducing protonation of absorption enhancers such as SNAC).

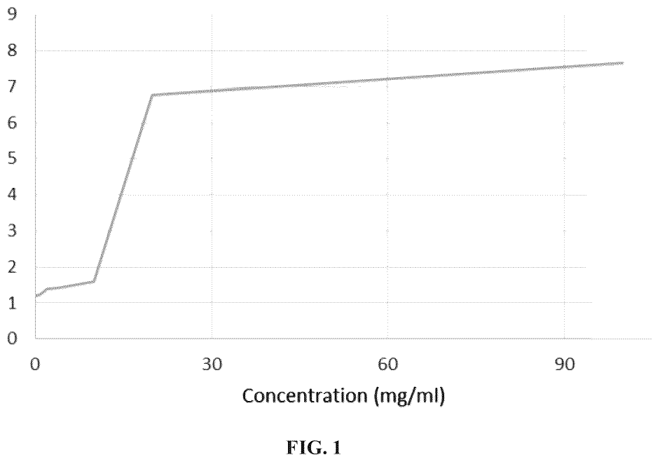

Referring now to the drawings, FIG. 1 shows that the exemplary absorption enhancer SNAC undergoes protonation upon contact with HCl (such as is present in stomach acid).

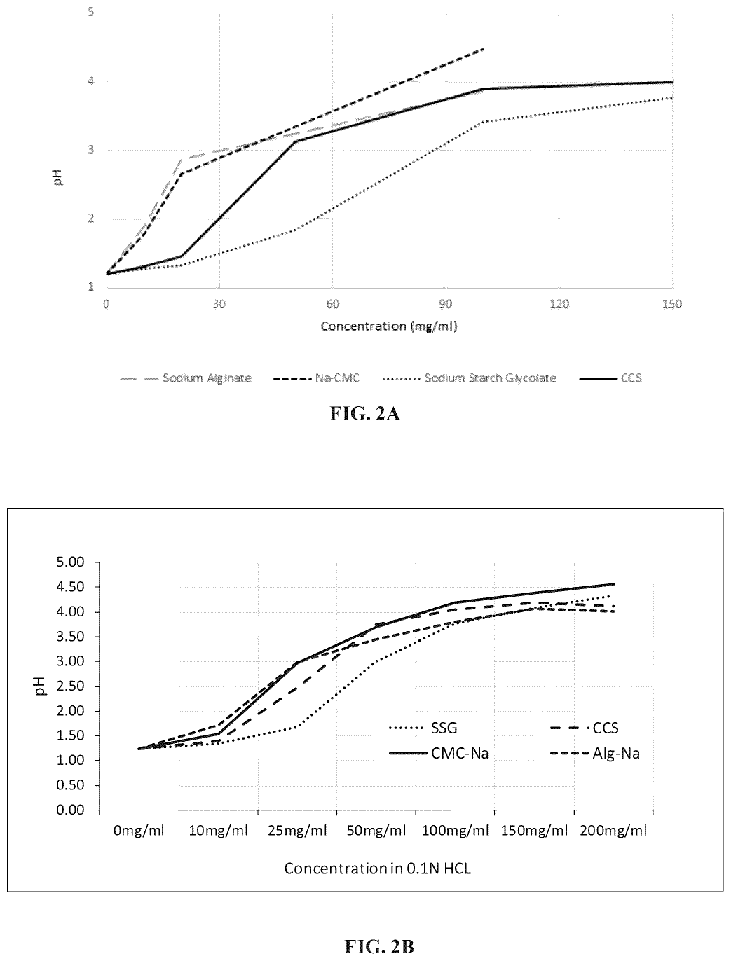

FIGS. 2A-B show that a variety of carboxylate group-containing polymers increase pH in a concentration-dependent manner.

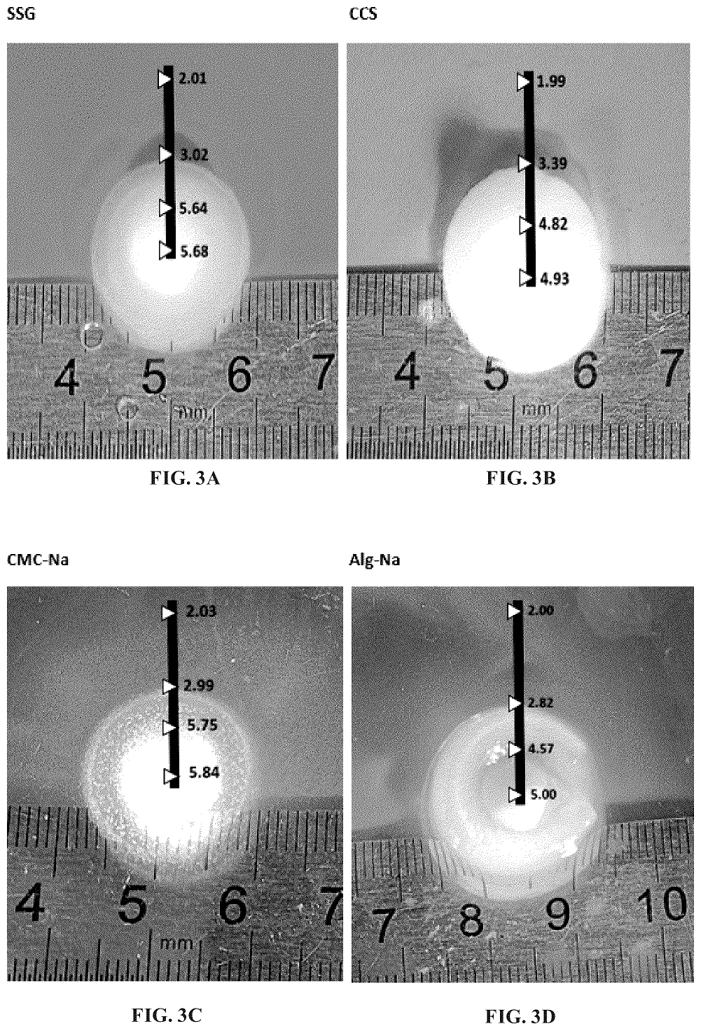

FIGS. 3A-D and 4A-B show the increased local pH inside tablets made of varying carboxylate group-containing polymers, upon hydration in acidic aqueous solutions.

FIG. 5 shows that exemplary carboxylate group-containing polymers considerably enhance absorption of a therapeutically active agent in in vivo models.

According to an aspect of some embodiments of the invention, there is provided a pharmaceutical composition comprising a therapeutically active agent, an absorption enhancer, and a polymer comprising a plurality of alkaline groups (which for brevity is also referred to herein as an “alkaline group-containing polymer” or “basic polymer” or simply “polymer”).

As explained in detail hereinbelow, the alkaline-group containing polymers is also referred to herein as an acid-neutralizing polymer.

According to the present embodiments, the therapeutically active agent is a glucagon-like peptide-2 (GLP-2) and/or a polypeptide which is a GLP-2 receptor agonist, as defined herein.

Herein, the terms “glucagon-like peptide-2” and “GLP-2” (which are used herein interchangeably) refer to a polypeptide (as defined herein) which is a naturally occurring GLP-2 or an analog (e.g., synthetic analog) thereof. The analog may be any derivative of the naturally occurring GLP-2, such as homologs (as defined herein), fragments (including fragments of a homolog) and substituted derivatives (e.g., comprising a substituent for enhancing stability and/or half-life) of the naturally occurring GLP-2 or homolog and/or fragment thereof, and/or be modified in any other way described herein with respect to polypeptides. Preferably, the analog exhibits a biological activity of the GLP-2.

Herein, the terms “glucagon-like peptide-2 receptor agonist” and “GLP-2 receptor agonist” (which are used herein interchangeably) refer to a polypeptide (as defined herein) which is a naturally occurring agonist of GLP-2 receptor (e.g., other than a GLP-2, as defined herein) or an analog (e.g., synthetic analog) thereof. The analog may be any derivative of the naturally occurring GLP-2 receptor agonist, such as homologs (as defined herein), fragments (including fragments of a homolog) and substituted derivatives (e.g., comprising a substituent for enhancing stability and/or half-life) of the naturally occurring GLP-2 receptor agonist or homolog and/or fragment thereof, and/or be modified in any other way described herein with respect to polypeptides. Preferably, the analog exhibits a biological activity of the GLP-2 receptor agonist.

In some of any of the embodiments described herein, the GLP-2 is teduglutide, apraglutide and/or glepaglutide. Teduglutide is an exemplary form of GLP-2.

In some of any of the embodiments described herein, the therapeutically active agent is a dual receptor agonist, for example, a dual GLP1/GLP2 receptor agonist. An exemplary such a dual receptor agonist is dapiglutide.

In some of any of the embodiments described herein, the therapeutically active agent is an analog of GLP-2 or of a GLP-2 receptor agonist or of a dual receptor agonist, which is long acting, namely, has enhanced plasma half-life, as, for example, in acetylated peptides or polypeptides.

As used herein, the singular form “a”, “an” and “the” include plural references unless the context clearly dictates otherwise. For example, the term “a polymer” or “at least one polymer” may include a plurality of polymers, including mixtures thereof.

In some of any of the embodiments described herein, the composition is in a form of a unit dosage form, for example, a tablet or a combination of more than one tablet (e.g., minitablets).

The term “unit dosage form”, as used herein, describes physically discrete units, each unit containing a predetermined quantity of one or more active ingredient(s) calculated to produce the desired therapeutic effect, in association with at least one pharmaceutically acceptable carrier, diluent, excipient, or combination thereof. Examples of a suitable unit dosage form include, without limitation, a tablet, a capsule, a lozenge, a dragee, a wafer, a sachet, a patch, an ampoule, a vial, a loaded syringe, and a dose of a metered-dose dispenser. Each unit may optionally comprise discrete subunits (e.g., wherein each subunit is in a form described herein, such as a tablet, capsule, lozenge, dragee, etc.), which may be attached to one another or separate from one another. For example, a unit dosage form according to embodiments of the invention which comprises multiple subunits may optionally be in a form of a unit dosage form comprising discrete subunits bound to one another by a coating and/or matrix, or in a form of a set of dosage forms in a kit (e.g., packaged sets of discrete dosage forms), as described in International Patent Application Publication No. WO 2018/033927, which is incorporated herein by reference.

The pharmaceutical composition and/or unit dosage form according to any of the respective embodiments described herein is preferably formulated to be suitable for oral administration, e.g., as described inmore detail herein. Examples of a unit dosage form formulated for oral administration include, without limitation, a tablet, a capsule, a lozenge, a dragee, a wafer, a sachet, an ampoule, and a vial. In exemplary embodiments, the pharmaceutical composition and/or unit dosage form according to any of the respective embodiments described herein is formulated as a tablet or as a plurality of minitablets. In exemplary embodiments, the pharmaceutical composition and/or unit dosage form according to any of the respective embodiments described herein is devoid of a gastroenteric coating, or otherwise comprises a coating that is dissolvable in gastric fluid and/or immediately (e.g., within less than 5 minutes, less than 2 minutes, or less than 1 minute) releases the composition's content upon contacting gastric fluid.

Herein, the phrases “oral administration”, “orally administering” and the like refer to any administration via the mouth, preferably by oral ingestion, such as by swallowing (e.g., as opposed to buccal administration).

In some of any of the embodiments described herein, the composition is formulated to allow absorption of the therapeutically active agent in the stomach, for example, by being devoid of an enteric coating, i.e., a coating which prevents release of an agent and dissolves only in the intestines.

Without being bound by any particular theory, it is believed that GLP-2 tends to be poorly absorbed upon oral administration, for example, due to their relatively large molecular weight and/or hydrophilicity (e.g., which inhibits crossing of lipid membranes in the gastrointestinal tract); and therefore, their absorption is particularly susceptible to enhancement by activity of an absorption enhancer (e.g., NAC or a salt thereof) and to increasing absorption enhancer activity by an alkaline group-containing polymer according to any of the respective embodiments described herein. It is further believed that absorption of such polypeptides may be further limited by stomach acid, for example, due to degradation by pH-dependent activity of enzymes such as pepsin, and that a pH increase associated with an alkaline group-containing polymer can enhance absorption of a polypeptide by reducing pepsin activity (in addition to increasing absorption enhancer activity, as described herein).

### Alkaline Group-Containing Polymer

Herein, the term “polymer” refers a compound having at least 4 repeating (backbone) units (and more preferably at least 10 repeating units, for example, from 4 to 1,000, or from 10 to 1,000 repeating units, with higher numbers of repeating units being also contemplated), the repeating units being identical or similar. The term “polymer” encompasses also a co-polymer, which comprises two or more types of repeating units as described herein, for example, at least 4, or preferably at least 10, repeating units of one type, and at least 4, or preferably at least 10, repeating units of another, different type. The units in a co-polymer can be arranged in any order.

The backbone (repeating) units composing a polymer or copolymer as described herein are also referred to herein interchangeably as monomeric units or simply as monomers.

The alkaline group-containing polymer comprises a plurality (i.e., at least 2) of alkaline groups per molecule. In some of any of the embodiments described herein, the alkaline group-containing polymer comprises at least 4 alkaline groups, or at least 10 alkaline groups, or at least 25 alkaline groups, or at least 50 alkaline groups, or at least 100 alkaline groups, on average, per molecule. In some of any of the embodiments described herein, an average number of the alkaline groups per polymer molecule ranges from about 4 to about 1,000, or from about 10 to about 1,000, or from about 25 to about 1,000, or from about 50 to about 1,000, or from about 100 to about 1,000, with higher upper limit also contemplated for each of these ranges.

In some of any of the embodiments described herein, a mean molecular weight of the alkaline group-containing polymer is at least 1 kDa, or at least 2 kDa, or at least 3 kDa, or at least 5 kDa, or at least 10 kDa. In some of any of the embodiments described herein, a mean molecular weight of the alkaline group-containing polymer ranges from about 1 kDa, or from about 2 kDa, or from about 3 kDa, or from about 5 kDa, or from about 10 kDa, or from about 100 kDa, or from about 200 kDa, or from about 300 kDa, or from about 400 kDa, or from about 500 kDa, or from about 600 kDa, or from about 700 kDa, or from about 800 kDa, or from about 1,000 kDa, or from about 2,000 kDa, and up to about 10,000 kDa or higher, including any intermediate values and subranges therebetween.

Herein, the term “alkaline group” encompasses any functional group capable of accepting a proton in aqueous solution, for example, carboxylate or amine groups. It will be appreciated that an alkaline group may be converted to an acidic group (by protonation), and vice versa (by deprotonation), upon contact with a liquid with an appropriate acidity or alkalinity, as these terms are used in the art (e.g., according to pH).

In some of any one of the respective embodiments described herein, the polymer is characterized by a pKa which is at least 1.2, optionally in a range of from 1.2 to 7.5, and optionally in a range of from 1.2 to 5.5 (including any intermediate values and subranges therebetween); for example, from 1.2 to 3 or from 2.5 to 3.5 or from 3 to 4 or from 3.5 to 4.5 or from 4 to 5 or from 4.5 to 5.5, including any intermediate values and subranges therebetween. In some such embodiments, the pKa is at least 2.5, or at least 3, or at least 3.5; for example, from 2.5 to 5.5, or from 3 to 5.5, or from 3.5 to 5.5, or from 4 to 5.5, or from 4.5 to 5.5, including any intermediate values and subranges therebetween.

Herein, the “pKa” of a polymer refers to the pH in an aqueous solution of 0.1 M NaCl, at which 50% of the acid/base (e.g., carboxylate) groups are protonated at 25° C.

Carboxylate groups are exemplary alkaline groups according to some embodiments of the invention.

Herein, the term “carboxylate” refers to —C(═O)O⁻ groups (i.e., a carboxylate anion), and does not encompass carboxylic acid groups (—C(═O)OH) unless specifically indicated. It will be appreciated that carboxylate groups may be converted to carboxylic acid groups, and vice versa, upon contact with a liquid with an appropriate acidity or alkalinity. For simplicity, some carboxylate group-containing compounds and functional groups described herein (e.g., carboxymethyl, polyacrylic acid) are named as if they were in the carboxylic acid form, but are to be understood as comprising carboxylate groups unless otherwise indicated.

Carboxylate groups according to any of the respective embodiments described herein may optionally be accompanied by a counter-ion to form a salt, for example, a pharmaceutically acceptable salt. A salt (pharmaceutically acceptable salt) of a compound as described herein can alternatively be formed during the synthesis of the compound, e.g., in the course of isolating the compound from a reaction mixture, or re-crystallizing the compound.

Examples of suitable counter-ions which may be comprised by a salt described herein include, without limitation, ammonium, guanidinium, lithium, sodium, potassium, calcium, and magnesium. Sodium is an exemplary counter-ion.

Examples of suitable carboxylate group-containing polymers include, without limitation, salts of alginate, polyacrylic acid, polymethacrylic acid and copolymers of acrylic acid and/or methacrylic acid, and carboxylate group-containing polysaccharide derivatives (e.g., derivatives obtained by oxidation or substitution by a carboxylate-containing group, such as carboxymethyl).

A carboxylate may optionally be comprised by a repeating monomer (backbone unit) which comprises carboxylate, such as a urinate form of a saccharide unit (e.g., mannuronate and guluronate monomers in alginate) and acrylate (e.g., as in a polyacrylic acid salt); comprised by a substituent (e.g., a carboxymethyl substituent, which may optionally be formed by reaction of a polymer with chloroacetic acid) attached to at least a portion of the monomers (e.g., as in carboxymethyl cellulose and sodium starch glycolate); and/or formed by oxidation of at least a portion of the monomers (backbone units), for example, by breaking carbon-carbon bonds and/or by oxidation of a primary carbon (e.g., as in oxidized starch). In some exemplary embodiments, the polymer comprises carboxymethyl groups attached to oxygen atoms.

An amine may optionally be comprised by a repeating monomer (backbone unit) which comprises the amine group; for example, an amino sugar such as a 2-amino-2-deoxysugar (e.g., glucosamine in chitosan), and alkylene imine (e.g., ethylene imine) residues (e.g., as in a polyethyleneimine); and/or comprised by a substituent.

The alkaline group-containing polymer may optionally contain monomers (backbone units) which do not comprise an alkaline group (e.g., acrylate esters, methacrylate esters, acrylamides and methacrylamides), for example, in a copolymer with acrylic acid or methacrylic acid monomers; and/or substituents which do not comprise an alkaline group (e.g., substituted or non-substituted alkyl groups such as methyl, ethyl, hydroxyethyl and hydroxypropyl), for example, in a polymer (e.g., polysaccharide) also substituted by an alkaline group-containing substituent.

The alkaline groups, upon oral administration, are such that are capable of neutralizing an acid by undergoing protonation, to form an acidic group (e.g., a carboxylic acid group or ammonium group), thus increasing the pH of a solution that is in contact with the respective alkaline group-containing polymer.

Without being bound by any particular theory, it is believed that the acid-neutralizing polymer described herein is more suitable than conventional antacids, as the polymer is a large molecule which does not readily diffuse throughout the stomach (thus minimizing dilution of the polymer), which may result in a longer period of efficacy. It is further believed that the polymer described herein provides an additional advantageous effect (which is generally lacking in conventional antacids) of forming a viscous medium which limits influx of stomach acid to the absorption enhancer, thus further reducing inactivation of absorption enhancer by stomach acid; and/or limits diffusion of the absorption enhancer and/or therapeutically active agent throughout the stomach, thus enhancing the effect of the absorption enhancer by maintaining a higher local concentration of absorption enhancer in the vicinity of the therapeutically active agent. In this manner the composition may facilitate absorption of the therapeutically effective agent for a longer time (e.g., by reducing the rate of inactivation of absorption enhancer by protonation and/or by dilution) and/or increase the peak rate of absorption of the therapeutically effective agent (e.g., by providing a higher local concentration of absorption enhancer in active form, as discussed herein). It is further believed that such formation of a viscous medium may facilitate adhesion of the composition to the stomach wall (e.g., by increasing contact area between the soft medium and the stomach wall), thereby promoting absorption via stomach tissue.

In some embodiments of any one of the embodiments described herein, the alkaline group-containing polymer swells upon contact with water (and is also referred to herein in the context of these embodiments as “water-swellable”). In some such embodiments, the polymer is not water-soluble.

Herein, the phrase “swells upon contact with water” refers to an ability of a substance to absorb at least its own weight in water upon contact with water (e.g., pure water) at 37° C. (that is, the substance has a swelling capacity of at least 100%), and optionally at least twice its own weight (a swelling capacity of at least 200%) or at least 5-fold its own weight (a swelling capacity of at least 500%) or at least 10-fold its own weight or at least 20-fold its own weight in water.

By “swelling capacity” it is meant that the material is capable of swelling the indicated weight percentage water, of its weight before swelling. The swelling capacity (Qt) can be calculated using the formula:

$$Qt = ( Ws - Wd ) / Ws * 100 \% .$$

wherein Ws is the weight after swelling and Wd is the dry weight (weight before swelling).

Herein, the term “water-soluble” refers to a compound having a solubility of at least 1 gram per liter in water (e.g., pure water) at 37° C.

Herein, the term “water-insoluble” refers to a compound having a solubility of less than 1 gram per liter in water (e.g., pure water) at 37° C.

A typical assay for quantitatively determining solubility of a substance is the “shake-flask method”, in which an excess of the tested substance added to a volume (e.g., 100 ml) of solvent (e.g., water) in a container (e.g., flask or vial) and shaken under predetermined conditions (e.g., a temperature of 37° C.) so as to achieve thermodynamic equilibrium. Residual solid is then removed (e.g., by filtration and/or centrifugation), and the concentration of dissolved substance is determined by a technique such as HPLC. The concentration may optionally be determined at different time points in order to ascertain that equilibrium has been reached.

Without being bound by any particular theory, it is believed that swelling upon contact with water (e.g., in gastric medium) is advantageous as it enhances penetration of water and maximizes contact between the surrounding solution and alkaline groups upon oral administration. For example, a polymer with a lower degree of penetration of water (e.g., into granules of the polymer) may exhibit slower than optimal neutralization of stomach acid, even if the amount of alkaline groups is high; for example, due to less effective diffusion of acid to the alkaline groups. It is further believed that water solubility, although also effective at maximizing contact between the surrounding solution and alkaline groups, is less desirable than swelling, as dissolution of the polymer disadvantageously results in a less localized effect (e.g., similar to conventional antacids discussed herein).

In some of any of the embodiments described herein, the polymer is a crosslinked polymer.

The polymer can be cross-linked via covalent bonds, by means of cross-linking moieties that are each being covalently attached to, and thereby connects, two or more backbone units in the polymer, and/or via electrostatic bonds, by means of cross-linking moieties (e.g., ions such as cations) that are each being electrostatically attached to, and thereby connects, two or more backbone units in the polymer. The number of cross-linking moieties determines the degree of cross-linking of a cross-linked polymer.

Without being bound by any particular theory, it is believed that crosslinking is advantageous by minimizing dissolution of the polymer (e.g., in saliva and/or stomach acid) which could result in “wastage” of the polymer; and/or by enhancing water-absorption (e.g., by disrupting binding between different polymer chains, thereby facilitating entry of water molecules between the chains). The degree of crosslinking may affect swelling capacity; for example, insufficient crosslinking may be associated with dissolution (as opposed to swelling), and/or to reduced swelling and/or reduced penetration of water. However, excessive crosslinking may reduce swelling capacity by limiting the ability of polymer to expand and provide space for water to enter. The degree of crosslinking should not affect the acid-neutralizing capacity of the polymer. The degree of cross-linking can be selected or pre-determined for each selected polymer, in accordance with its properties, so as to provide the desired hydration level.

In some of any of the embodiments described herein, the polymer comprises a polysaccharide, for example, a crosslinked polysaccharide. The polymer is optionally composed primarily (i.e., more than 50 percent by weight) of glucose units, which may optionally be linked, by glycosidic bonds such as α(1→4) and/or α(1→6) glycosidic bonds (e.g., as in starch) and/or β(1→4) glycosidic bonds (e.g., as in cellulose).

The term “monosaccharide”, as used herein and is well known in the art, refers to a simple form of a sugar that consists of a single saccharide molecule which cannot be further decomposed by hydrolysis. Most common examples of monosaccharides include glucose (dextrose), fructose, galactose, and ribose. Monosaccharides can be classified according to the number of carbon atoms of the carbohydrate, i.e., triose, having 3 carbon atoms such as glyceraldehyde and dihydroxyacetone; tetrose, having 4 carbon atoms such as erythrose, threose and erythrulose; pentose, having 5 carbon atoms such as arabinose, lyxose, ribose, xylose, ribulose and xylulose; hexose, having 6 carbon atoms such as allose, altrose, galactose, glucose, gulose, idose, mannose, talose, fructose, psicose, sorbose and tagatose; heptose, having 7 carbon atoms such as mannoheptulose, sedoheptulose; octose, having 8 carbon atoms such as 2-keto-3-deoxy-manno-octonate; nonose, having 9 carbon atoms such as sialose; and decose, having 10 carbon atoms.

Monosaccharides are the building blocks of polysaccharides (such as cellulose and starch).

The term “polysaccharide” as used herein refers to a compound that comprises 10 or more monosaccharide units, as these are defined herein, linked to one another via a glycosyl bond (—O—). The glycosyl bonds between saccharide units in a polysaccharide can all be the same, or can include two or more types of glycosyl bonds, for example, be such that glucose units are linked via α(1→4) and/or α(1→6) glycosidic bonds.

Herein, the term “starch” encompasses polysaccharides composed of amylose (glucose units linked via α(1→4) glycosidic bonds), and amylopectin (glucose units linked via α(1→4) and α(1→6) glycosidic bonds). Different types of starch differ from one another by the ratio between amylose and amylopectin, which typically depends on the source of the starch.

In some of any of the embodiments relating to a polysaccharide, the polysaccharide is a starch derivative and/or a cellulose derivative, that is, starch and/or cellulose (e.g., crosslinked starch and/or crosslinked cellulose) derivatized so as to comprise alkaline (e.g., carboxylate) groups, for example, by being substituted by carboxymethyl groups (e.g., wherein oxygen atoms thereof are attached to carboxymethyl groups). Sodium starch glycolate is an exemplary starch derivative. Croscarmellose sodium is an exemplary cellulose derivative.

Herein, “sodium starch glycolate” refers to any sodium salt of starch (as defined herein) substituted by carboxymethyl groups, and may be crosslinked or non-crosslinked. In some of any of the respective embodiments, the sodium starch glycolate is crosslinked covalently by phosphate groups (—O—P(═O)(—O⁻)—O—), each linking two (or more) saccharide (glucose) units.

Herein, “croscarmellose sodium” refers to any sodium salt of cellulose (as defined herein) substituted by carboxymethyl groups, and crosslinked covalently by ester bonds between carboxymethyl groups and a saccharide unit.

The carboxymethyl groups (or any other alkaline group(s)) can be attached to one or more of the hydroxy substituents of one or more saccharide (e.g., glucose) units composing the polysaccharide, so as to form a —O═CH₂—C(═O)—O⁻ alkaline group which substitute the monosaccharide at one or more positions. In some embodiments, the carboxymethyl groups (or any other alkaline group(s)) are attached at least to the hydroxymethyl group of one or more glucose units that compose the polysaccharide. In some embodiments, the carboxymethyl groups (or any other alkaline group(s)) are attached to at least 10%, or at least 20%, or at least 30%, or at least 40%, or at least 50%, or at least 60%, or at least 70%, or at least 80%, or at least 90% or about all of the saccharide units composing the polysaccharide. In some of these embodiments, one carboxymethyl group (or any other alkaline group) is attached to a saccharide unit comprising same; and in some of these embodiments, the carboxymethyl group (or any other alkaline group) is attached to the hydroxymethyl substituent of the glucose.

In some of any of the embodiments described herein, a concentration of the polymer comprising a plurality of alkaline groups (according to any of the respective embodiments described herein) in the composition is at least 10 weight percent. In some such embodiments, the concentration of the polymer is at least 15 weight percent. In some embodiments, the In some embodiments, the concentration of the polymer is at least 20 weight percent. concentration of the polymer is at least 25 weight percent. In some embodiments, the In some embodiments, the concentration of the polymer is at least 30 weight percent. concentration of the polymer is at least 35 weight percent. In some embodiments, the concentration of the polymer is at least 40 weight percent. In some embodiments, the concentration of the polymer is at least 50 weight percent. In some embodiments, the concentration of the polymer is at least 60 weight percent. In some embodiments, the concentration of the polymer is at least 70 weight percent. In some embodiments, the concentration of the polymer is at least 80 weight percent. In some embodiments, the concentration of the polymer is at least 90 weight percent.

By “weight percent” (or w/w %, or % wt.) it is meant the weight of the indicated substance out of the total weight of a composition comprising same.

In some of any of the embodiments described herein, a concentration of the polymer comprising a plurality of alkaline groups (according to any of the respective embodiments described herein) in the composition ranges from 10 to 90 weight percent, of the total weight of the composition, or from 10 to 80, or from 10 to 70, or from 10 to 50, or from 10 to 40, or from 10 to 30, or from 20 to 90, or from 20 to 80, or from 20 to 70, or from 20 to 60, or from 20 to 50, or from 20 to 40, or from 20 to 30, or from 30 to 90, or from 30 to 80, or from 30 to 70, or from 30 to 60, or from 30 to 50, or from 30 to 40, or from 40 to 90, or from 40 to 80, or from 40 to 70, or from 40 to 60, or from 40 to 50, or from 50 to 90, or from 50 to 80, or from 50 to 70, or from 50 to 60, or from 60 to 90, or from 60 to 80, or from 60 to 70, or from 80 to 90, or from 70 to 80, or from 80 to 90, weight percent, of the total weight of the composition, including any intermediate values and subranges therebetween.

Without being bound by any particular theory, it is believed that a relatively high concentration of water-absorbing (e.g., water-swellable) polymers such as sodium starch glycolate and croscarmellose sodium (e.g., relative to compositions which utilize such polymers as disintegrants) is associated with generation of a viscous medium upon contact with an aqueous solution (e.g., stomach acid) which limits diffusion in the vicinity of the composition, which is significantly different than their effect (e.g., promotion of disintegration of a solid composition) at lower concentrations.

Compositions which utilize such polymers as disintegrants further differ from compositions according to some embodiments of the invention in that disintegrants are normally used in compositions which also comprise a large proportion of insoluble components such as microcrystalline cellulose (e.g., as opposed to compositions comprising large proportions of water soluble components such as SNAC); for example, because the mechanism of the disintegrant involves applying internal forces to a solid insoluble component and/or because a large proportion of water soluble component(s) renders a composition readily soluble, such that a disintegrant would be redundant.

In some of any of the embodiments relating to a polysaccharide described herein, the polysaccharide is characterized by a degree of substitution which is at least 0.1; for example, in a range of from 0.1 to 2 or from 0.1 to 1.5 or from 0.1 to 1 or from 0.1 to 0.5 or from 0.1 to 0.3 or from 0.1 to 0.2, including any intermediate values and subranges therebetween. In some such embodiments, the degree of substitution is at least 0.2; for example, in a range of from 0.2 to 2 or from 0.2 to 1.5 or from 0.2 to 1 or from 0.2 to 0.5, including any intermediate values and subranges therebetween. In some embodiments, the degree of substitution is at least 0.3; for example, in a range of from 0.3 to 2 or from 0.3 to 1.5 or from 0.3 to 1 or from 0.3 to 0.5, including any intermediate values and subranges therebetween. In some embodiments, the degree of substitution is at least 0.4; for example, in a range of from 0.4 to 2 or from 0.4 to 1.5 or from 0.4 to 1, including any intermediate values and subranges therebetween.

Herein, “degree of substitution” refers to the ratio of functional groups comprising an alkaline group or a conjugate acid thereof, such as a carboxylate or carboxylic acid group (e.g., a carboxymethyl group), to monomers (backbone units) of a polymer (e.g., saccharide units of a polysaccharide).

In some of any of the respective embodiments described herein, a concentration of alkaline groups of the polymer in the composition is at least 0.03 millimoles per gram composition; for example, from 0.03 to 5 millimoles per gram or from 0.03 to 2 millimoles per gram or from 0.03 to 1 millimoles per gram or from 0.03 to 0.5 millimoles per gram or from 0.03 to 0.2 millimoles per gram, including any intermediate values and subranges therebetween. In some embodiments, the concentration of alkaline groups of the polymer is at least 0.1 millimoles per gram composition; for example, from 0.1 to 5 millimoles per gram or from 0.1 to 2 millimoles per gram or from 0.1 to 1 millimoles per gram or from 0.1 to 0.5 millimoles per gram, including any intermediate values and subranges therebetween. In some embodiments, the concentration of alkaline groups of the polymer is at least 0.2 millimoles per gram composition; for example, from 0.2 to 5 millimoles per gram or from 0.2 to 2 millimoles per gram or from 0.2 to 1 millimoles per gram or from 0.2 to 0.5 millimoles per gram, including any intermediate values and subranges therebetween. In some embodiments, the concentration of alkaline groups of the polymer is at least 0.5 millimoles per gram composition; for example, from 0.5 to 5 millimoles per gram or from 0.5 to 2 millimoles per gram or from 0.5 to 1 millimoles per gram, including any intermediate values and subranges therebetween. In some embodiments, the concentration of alkaline groups of the polymer is at least 1 millimoles per gram composition; for example, from 1 to 5 millimoles per gram or from 1 to 2 millimoles per gram, including any intermediate values and subranges therebetween.

In some of any of the embodiments described herein, the composition is a unit dosage form composition, and an amount of alkaline groups of the polymer in the unit dosage form (which correlates with the acid-neutralizing capacity of the polymer in the unit dosage form) is at least 0.03 millimoles; for example, from 0.03 to 10 millimoles or from 0.03 to 3 millimoles or from 0.03 to 1 millimoles or from 0.03 to 0.3 millimoles, including any intermediate values and subranges therebetween. In some embodiments, the amount of alkaline groups in the unit dosage form is at least 0.1 millimoles; for example, from 0.1 to 10 millimoles or from 0.1 to 3 millimoles or from 0.1 to 1 millimoles, including any intermediate values and subranges therebetween. In some embodiments, the amount of alkaline groups in the unit dosage form is at least 0.3 millimoles; for example, from 0.3 to 10 millimoles or from 0.3 to 3 millimoles, including any intermediate values and subranges therebetween. In some embodiments, the amount of alkaline groups in the unit dosage form is at least 1 millimoles; for example, from 1 to 10 millimoles, including any intermediate values and subranges therebetween.

As mentioned hereinabove, the alkaline group-containing polymer according to any of the respective embodiments described herein may optionally comprise two or more (e.g., at least 3 or at least 4) distinct polymers (e.g., in admixture) which comprise alkaline groups. Thus, for example, sodium starch glycolate may represent a portion of all the alkaline group-containing polymer, e.g., at least 10 weight percent or at least 20 weight percent or at least 30 weight percent or at least 40 weight percent or at least 50 weight percent or at least 60 weight percent or at least 70 weight percent or at least 80 weight percent or at least 90 weight percent of the alkaline group-containing polymer (according to any of the respective embodiments described herein), the remainder being one or more alkaline group-containing polymers other than sodium starch glycolate. Alternatively or additionally, croscarmellose sodium may represent a portion of all the alkaline group-containing polymer, e.g., at least 10 weight percent or at least 20 weight percent or at least 30 weight percent or at least 40 weight percent or at least 50 weight percent or at least 60 weight percent or at least 70 weight percent or at least 80 weight percent or at least 90 weight percent of the alkaline group-containing polymer (according to any of the respective embodiments described herein), the remainder being one or more alkaline group-containing polymers other than croscarmellose sodium.

It is noted that polymers in general typically comprise a population of molecules of different sizes and with slightly different geometries (e.g., branching patterns and/or sequence of monomers in a copolymer). In accordance with standard practice in the art, such a population of molecules is not considered to represent “distinct polymers”. Rather, “distinct polymers” refers to chemical differences, such as polymers composed of different monomers (backbone units) and/or crosslinkers and/or of different degree of cross-linking.

The effect of the polymer on absorption of the therapeutically active agent may optionally be determined by comparing absorption upon oral administration of a composition according to embodiments of the invention with a corresponding composition comprising the same amount of other ingredients other than the alkaline group-containing polymer (e.g., when the alkaline group-containing polymer is no more than 50 weight percent of the composition according to embodiments of the invention), such that the total mass is lower (due to the absence of alkaline group-containing polymer). In some such embodiments, both compositions are in a form of a tablet, and the tablets have the same cross-section (e.g., diameter of a circular cross-section), and the tablet with a lower weight is thinner in the axis perpendicular to the cross-section of the tablet.

Alternatively, the effect of the polymer on absorption of the therapeutically active agent may optionally be determined by comparing absorption upon oral administration of a composition according to embodiments of the invention with a corresponding composition comprising absorption enhancer instead of the alkaline group-containing polymer (e.g., the corresponding composition being identical in all aspects except for the presence of additional absorption enhancer instead of the polymer), such that the total mass of the compositions is the same (e.g., when the alkaline group-containing polymer is more than 50 weight percent of the composition according to embodiments of the invention).

In some embodiments of any one of the embodiments described herein, a Cmax and/or bioavailability of the composition (i.e., of the therapeutically active agent in the composition) upon oral administration is at least 20% higher than (120% of the level of) a Cmax and/or bioavailability of a corresponding composition without the alkaline group-containing polymer. In some embodiments, the Cmax and/or bioavailability is at least 50% higher than (150% of the level of) the Cmax and/or bioavailability upon oral administration of a corresponding composition without the alkaline group-containing polymer. In some embodiments, the Cmax and/or bioavailabilityis at least twice (200% of the level of) the Cmax and/or bioavailability upon oral administration of a corresponding composition without the alkaline group-containing polymer. In some embodiments, the Cmax and/or bioavailability is at least four-fold (400% of the level of) the Cmax and/or bioavailability upon oral administration of a corresponding composition without the alkaline group-containing polymer. In some embodiments, the Cmax and/or bioavailability is at least six-fold (600% of the level of) the Cmax and/or bioavailability upon oral administration of a corresponding composition without the alkaline group-containing polymer. In some embodiments, the Cmax and/or bioavailability is at least ten-fold (1000% of the level of) the Cmax and/or bioavailability upon oral administration of a corresponding composition without the alkaline group-containing polymer.

Cmax and/or bioavailability may optionally be determined by administering the composition orally to subjects (e.g., human subjects) and determining a level of the therapeutically active agent in the blood at frequent intervals by taking blood samples.

Bioavailability may be determined by comparing a ratio of an area under the curve upon oral administration (e.g., over the course of 12 or 24 hours) to an area under curve over the same time period for intravenously administered therapeutically active agent, using standard techniques (e.g., data processing algorithms) known in the art. The therapeutically active agent may be injected at a lower dose (e.g., for safety reasons) and the areas under curve normalized to the total amount administered.

### Absorption Enhancer

According to preferred embodiments of any of the embodiments described herein, the composition according to any of the respective embodiments described herein comprises an effective amount of an absorption enhancer, i.e., an amount of absorption enhancer effective for enhancing absorption of the therapeutically active agent in the composition.

Herein, the term “absorption enhancer” refers to a compound known to enhance absorption of macromolecular drugs (e.g., compounds having a molecular weight of at least 1 kDa) from the gastrointestinal tract into the circulation upon oral administration of the drug. The person skilled in the art will be aware of many such absorption enhancers.

In some of any of the embodiments described herein, the absorption enhancer is a fatty acid, optionally with a terminal N-(2-hydroxybenzoyl)amino group (at the omega position, i.e., the terminus distal from the carboxylate group of the fatty acid), or a salt thereof (e.g., a monosodium or disodium salt). Alternatively, the fatty acid may optionally be a non-substituted fatty acid (e.g., caproic acid, caprylic acid, capric acid, lauric acid, oleic acid and/or stearic acid).

The fatty acid (substituted or non-substituted) is preferably from 4 to 20 carbon atoms in length, optionally from 4 to 18 carbon atoms in length, optionally from 4 to 16 carbon atoms in length, optionally from 4 to 14 carbon atoms in length, optionally from 4 to 12 carbon atoms in length and optionally from 4 to 10 carbon atoms in length, including any intermediate values and subranges therebetween. In some of any of the embodiments described herein, the fatty acid is from 6 to 20 carbon atoms in length, optionally from 6 to 18 carbon atoms in length, optionally from 6 to 16 carbon atoms in length, optionally from 6 to 14 carbon atoms in length, optionally from 6 to 12 carbon atoms in length, optionally from 6 to 10 carbon atoms in length, and optionally from 8 to 10 carbon atoms in length, including any intermediate values and subranges therebetween. The fatty acid moiety may be saturated (e.g., as are caprylic acid in 8-N-(2-hydroxybenzoyl)aminocaprylic acid and decanoic acid in 10-N-(2-hydroxybenzoyl)aminodecanoic acid) or unsaturated (i.e., comprising at least one unsaturated carbon-carbon bond).

Examples of suitable fatty acids (e.g., for substitution by a terminal N-(2-hydroxybenzoyl)amino group) include, without limitation, butanoic acid, caprylic acid and decanoic acid.

The N-(2-hydroxybenzoyl)amino group may optionally be substituted or non-substituted (e.g., on the aromatic ring thereof). Suitable substituents include, for example, halo (optionally chloro) and alkoxy (optionally methoxy). Examples of substituted N-(2-hydroxybenzoyl)amino groups include, without limitation, N-(5-chlorosalicyloyl)amino, N-(4-chloro-2-hydroxybenzoyl)amino, and N-(2-hydroxy-4-methoxybenzoyl)amino.

Examples of suitable absorption enhancers include, without limitation, NAC (8-N-(2-hydroxybenzoyl)aminocaprylic acid) and NAD (10-N-(2-hydroxybenzoyl)aminodecanoic acid) and salts thereof (e.g., monosodium and disodium salts); as well as derivatives thereof (e.g., derivatives substituted by chloro and/or methoxy) such as 5-CNAC (8-N-(5-chlorosalicyloyl)aminocaprylic acid) and 4-MOAC (8-N-(2-hydroxy-4-methoxybenzoyl)aminocaprylic acid) and salts thereof (e.g., monosodium and disodium salts). 4-CNAB (4-N-(2-hydroxy-4-chlorobenzoyl)aminobutanoic acid) and salts thereof (e.g., monosodium and disodium salts) are additional examples of a suitable absorption enhancer.

In some of any of the embodiments described herein, the absorption enhancer is in a form of a salt thereof, for example, a sodium salt. In exemplary embodiments, the sodium salt is a monosodium salt.

In some of any of the embodiments described herein, the absorption enhancer is NAC or NAD, or a salt thereof. In some such embodiments, the absorption enhancer is NAC or a salt thereof.

As shown below, the structure of NAD (depicted as a sodium salt thereof, also referred to as “SNAD”) differs from that of NAC (depicted as a sodium salt thereof, also referred to as “SNAC”) only in the length of the fatty acid moiety. Additional absorption enhancers related to NAC and NAD, based on different fatty acid lengths, will be readily apparent to the skilled person.

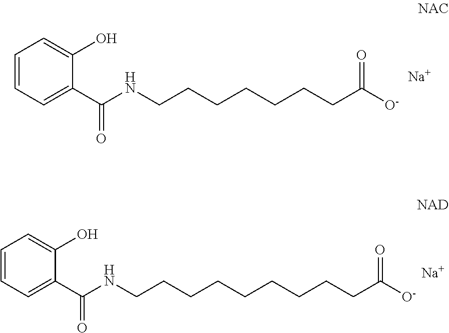 [SMILES: O=C([O-])CCCCCCCNC(=O)c1ccccc1O ; [Na+] ; O=C([O-])CCCCCCCCCNC(=O)c1ccccc1O ; [Na+]]

Without being bound by any particular theory, it is believed that many absorption enhancers tend to be more active (at enhancing absorption) in an ionic form (e.g., a carboxylate anion, such as a fatty acid anion) than in a less soluble non-ionic form (e.g., a carboxylic acid, such as a fatty acid), and that overall activity of the absorption enhancer is strongly dependent on concentration of the more active form. For example, the activity of the less soluble non-ionic form may be reduced by precipitation. It is further believed that the amount of absorption enhancer in ionic form upon dissolution following oral administration may be enhanced by providing the absorption enhancer in a form of a salt and/or by controlling a local pH in the vicinity of the absorption enhancer upon oral administration (e.g., by acid neutralization by alkaline groups of a polymer described herein and/or even by other molecules of absorption enhancer).

According to some of any of the embodiments described herein, the absorption enhancer is an ionizable material (compound), and in some of these embodiments, the absorption enhancer is more active in its ionic form.

According to some of any of the embodiments described herein, the absorption enhancer is inactivated by stomach acid, as described herein, and in some of these embodiments, the absorption enhancer is ionizable, is more active in its ionic form, and at least a portion of the absorption enhancer is in a non-ionic (less active) form when contacted with stomach acid (due, e.g., to precipitation).

It is further believed that formation of a viscous medium may enhance the effect of absorption enhancers which are not inactivated by stomach acid, e.g., by maintaining a higher local concentration of absorption enhancer in the vicinity of the therapeutically active agent and/or facilitating adhesion of the composition to the stomach wall (e.g., as described herein).

Additionally or alternatively, the control over local pH associated with the polymer may protect the therapeutically active agent from inactivation by stomach acid (regardless of whether the absorption enhancer is inactivated by stomach acid), for example, inactivation associated with pepsin activity in the presence of a suitably acidic pH (e.g., as discussed elsewhere herein).

According to some of any of the embodiments described herein, the absorption enhancer is not inactivated by stomach acid, for example, it is not ionizable and/or its ionic form and its non-ionic forms exhibit the same activity and/or it is not converted to a less active form when contacted with stomach acid (for example, the absorption enhancer is acidic by itself).

Examples of absorption enhancers which are not expected to be inactivated by stomach acid include, without limitation, alkyl fatty acid esters (e.g., isopropyl myristate); phospholipids (e.g., phosphatidyl choline); quaternary ammonium salts, such as tetraalkyl ammonium salts (e.g., cetyltrimethylammonium salts, such as cetyltrimethylammonium bromide) and alkyl pyridinium salts (e.g., cetylpyridinium salts such as cetylpyridinium chloride); nonionic surfactants such as sorbitan-fatty acid esters (e.g., sorbitan monolaurate, sorbitan monostearate, sorbitan tristearate) and polysorbates (i.e., ethoxylated sorbitan-fatty acid esters, e.g., polysorbate 20, polysorbate 40, polysorbate 60 and polysorbate 80); and N-dodecylcaprolactam.

In some of any one of the embodiments described herein, a concentration of the absorption enhancer (according to any of the respective embodiments described herein) in a composition (according to any of the respective embodiments described herein) is at least 10 weight percent; for example, from 10 to 90 weight percent, or from 10 to 80 weight percent, or from 10 to 70 weight percent, or from 10 to 60 weight percent, or from 10 to 50 weight percent, or from 10 to 40 weight percent, or from 10 to 30 weight percent, including any intermediate values and subranges therebetween. In some such embodiments, the concentration of the absorption enhancer is at least 20 weight percent; for example, from 20 to 90 weight percent, or from 20 to 80 weight percent, or from 20 to 70 weight percent, or from 20 to 60 weight percent, or from 20 to 50 weight percent, or from 20 to 40 weight percent, including any intermediate values and subranges therebetween. In some embodiments, the concentration of the absorption enhancer is at least 30 weight percent; for example, from 30 to 90 weight percent, or from 30 to 80 weight percent, or from 30 to 70 weight percent, or from 30 to 60 weight percent, or from 30 to 50 weight percent, including any intermediate values and subranges therebetween. In some embodiments, the concentration of the absorption enhancer is at least 40 weight percent; for example, from 40 to 90 weight percent, or from 40 to 80 weight percent, or from 40 to 70 weight percent, or from 40 to 60 weight percent, including any intermediate values and subranges therebetween. In some embodiments, the concentration of the absorption enhancer is at least 50 weight percent; for example, from 50 to 90 weight percent, or from 50 to 80 weight percent, or from 50 to 70 weight percent, including any intermediate values and subranges therebetween. In some embodiments, the concentration of the absorption enhancer is at least 60 weight percent; for example, from 60 to 90 weight percent, or from 60 to 80 weight percent, including any intermediate values and subranges therebetween. In some embodiments, the concentration of the absorption enhancer is at least 70 weight percent; for example, from 70 to 90 weight percent or from 70 to 80 weight percent, including any intermediate values and subranges therebetween. In some embodiments, the concentration of the absorption enhancer is at least 80 weight percent; for example, from 80 to 90 weight percent, including any intermediate values and subranges therebetween.

In some of any of the aforementioned embodiments, the absorption enhancer is NAC, NAD, 5-CNAC, 4-MOAC and/or 4-CNAB, or a salt thereof (e.g., a sodium salt thereof).

In some of any one of the embodiments described herein, a total concentration of the absorption enhancer (according to any of the respective embodiments described herein) and the polymer comprising the alkaline groups (according to any of the respective embodiments described herein) is at least 80 weight percent (e.g., from 80 to 100 weight percent, including any intermediate values and subranges therebetween). In some such embodiments, the concentration of the absorption enhancer is at least 10 weight percent or at least 20 weight percent or at least 30 weight percent or at least 40 weight percent or at least 50 weight percent or at least 60 weight percent or at least 70 weight percent (e.g., according to any of the respective embodiments described herein). Alternatively or additionally, in some embodiments, the concentration of the polymer comprising the alkaline groups is at least 10 weight percent or at least 20 weight percent or at least 30 weight percent or at least 40 weight percent or at least 50 weight percent or at least 60 weight percent or at least 70 weight percent (e.g., according to any of the respective embodiments described herein). In some of any of the aforementioned embodiments, the absorption enhancer is NAC, NAD, 5-CNAC, 4-MOAC and/or 4-CNAB, or a salt thereof (e.g., a sodium salt thereof).

In some of any one of the embodiments described herein, a total concentration of the absorption enhancer (according to any of the respective embodiments described herein) and the polymer comprising the alkaline groups (according to any of the respective embodiments described herein) is at least 90 weight percent (e.g., from 90 to 100 weight percent, including any intermediate values and subranges therebetween). In some such embodiments, the concentration of the absorption enhancer is at least 10 weight percent or at least 20 weight percent or at least 30 weight percent or at least 40 weight percent or at least 50 weight percent or at least 60 weight percent or at least 70 weight percent or at least 80 weight percent (e.g., according to any of the respective embodiments described herein). Alternatively or additionally, in some embodiments, the concentration of the polymer comprising the alkaline groups is at least 10 weight percent or at least 20 weight percent or at least 30 weight percent or at least 40 weight percent or at least 50 weight percent or at least 60 weight percent or at least 70 weight percent or at least 80 weight percent (e.g., according to any of the respective embodiments described herein). In some of any of the aforementioned embodiments, the absorption enhancer is NAC, NAD, 5-CNAC, 4-MOAC and/or 4-CNAB, or a salt thereof (e.g., a sodium salt thereof).

In some of any one of the embodiments described herein, a total concentration of the absorption enhancer (according to any of the respective embodiments described herein) and the polymer comprising alkaline groups (according to any of the respective embodiments described herein) is at least 95 weight percent (e.g., from 95 to 100 weight percent, including any intermediate values and subranges therebetween). In some such embodiments, the concentration of the absorption enhancer is at least 10 weight percent or at least 20 weight percent or at least 30 weight percent or at least 40 weight percent or at least 50 weight percent or at least 60 weight percent or at least 70 weight percent or at least 80 weight percent (e.g., according to any of the respective embodiments described herein). Alternatively or additionally, in some embodiments, the concentration of the polymer comprising the alkaline groups is at least 10 weight percent or at least 20 weight percent or at least 30 weight percent or at least 40 weight percent or at least 50 weight percent or at least 60 weight percent or at least 70 weight percent or at least 80 weight percent (e.g., according to any of the respective embodiments described herein). In some of any of the aforementioned embodiments, the absorption enhancer is NAC, NAD, 5-CNAC, 4-MOAC and/or 4-CNAB, or a salt thereof (e.g., a sodium salt thereof).

In some of any one of the embodiments described herein, a total concentration of the absorption enhancer (according to any of the respective embodiments described herein) and the polymer comprising alkaline groups (according to any of the respective embodiments described herein) is at least 98 weight percent. In some such embodiments, the concentration of the absorption enhancer is at least 10 weight percent or at least 20 weight percent or at least 30 weight percent or at least 40 weight percent or at least 50 weight percent or at least 60 weight percent or at least 70 weight percent or at least 80 weight percent (e.g., according to any of the respective embodiments described herein). In some embodiments, the concentration of the polymer comprising the alkaline groups is at least 10 weight percent or at least 20 weight percent or at least 30 weight percent or at least 40 weight percent or at least 50 weight percent or at least 60 weight percent or at least 70 weight percent or at least 80 weight percent (e.g., according to any of the respective embodiments described herein). In some of any of the aforementioned embodiments, the absorption enhancer is NAC, NAD, 5-CNAC, 4-MOAC and/or 4-CNAB, or a salt thereof (e.g., a sodium salt thereof).

In some of any one of the embodiments relating to a unit dosage form described herein, an amount of the absorption enhancer in the unit dosage form is at least 25 mg; for example, from 25 to 1000 mg or from 25 to 500 mg or from 25 to 250 mg or from 25 to 100 mg or from 25 to 50 mg, including any intermediate values and subranges therebetween. In some embodiments, the amount of the absorption enhancer in the unit dosage form is at least 50 mg; for example, from 50 to 1000 mg or from 50 to 500 mg or from 50 to 250 mg or from 50 to 100 mg, including any intermediate values and subranges therebetween. In some embodiments, the amount of the absorption enhancer in the unit dosage form is at least 75 mg; for example, from 75 to 1000 mg or from 75 to 500 mg or from 75 to 250 mg, including any intermediate values and subranges therebetween. In some embodiments, the amount of the absorption enhancer in the unit dosage form is at least 100 mg;

for example, from 100 to 1000 mg or from 100 to 500 mg or from 100 to 250 mg, including any intermediate values and subranges therebetween. In some embodiments, the amount of the absorption enhancer in the unit dosage form is at least 150 mg; for example, from 150 to 1000 mg or from 150 to 500 mg or from 150 to 250 mg, including any intermediate values and subranges therebetween. In some embodiments, the amount of the absorption enhancer in the unit dosage form is at least 200 mg; for example, from 200 to 1000 mg or from 200 to 500 mg, including any intermediate values and subranges therebetween. In some embodiments, the amount of the absorption enhancer in the unit dosage form is at least 300 mg; for example, from 300 to 1000 mg or from 300 to 500 mg, including any intermediate values and subranges therebetween. In some of any of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to the therapeutically active agent in the composition is at least 1:1 (absorption enhancer: therapeutically active agent), optionally in a range of from 1:1 to 1000:1, or from 1:1 to 500:1, or from 1:1 to 300:1, or from 1:1 to 200:1, or from 1:1 to 100:1, or from 1:1 to 50:1, or from 1:1 to 30:1, or from 1:1 to 20:1, or from 1:1 to 10:1, or from 1:1 to 5:1, or from 1:1 to 3:1, or from 1:1 to 2:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to the therapeutically active agent in the composition is at least 2:1 (absorption enhancer: therapeutically active agent), optionally in a range of from 2:1 to 1000:1, or from 2:1 to 500:1, or from 2:1 to 300:1, or from 2:1 to 200:1, or from 2:1 to 100:1, or from 2:1 to 50:1, or from 2:1 to 30:1, or from 2:1 to 20:1, or from 2:1 to 10:1, or from 2:1 to 5:1, or from 2:1 to 3:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to the therapeutically active agent in the composition is at least 3:1 (absorption enhancer: therapeutically active agent), optionally in a range of from 3:1 to 1000:1, or from 3:1 to 500:1, or from 3:1 to 300:1, or from 3:1 to 200:1, or from 3:1 to 100:1, or from 3:1 to 50:1, or from 3:1 to 30:1, or from 3:1 to 20:1, or from 3:1 to 10:1, or from 3:1 to 5:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to the therapeutically active agent in the composition is at least 5:1 (absorption enhancer: therapeutically active agent), optionally in a range of from 5:1 to 1000:1, or from 5:1 to 500:1, or from 5:1 to 300:1, or from 5:1 to 200:1, or from 5:1 to 100:1, or from 5:1 to 50:1, or from 5:1 to 30:1, or from 5:1 to 20:1, or from 5:1 to 10:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to therapeutically active agent in the composition is at least 10:1 (absorption enhancer: therapeutically active agent), optionally in a range of from 10:1 to 1000:1, or from 10:1 to 500:1, or from 10:1 to 300:1, or from 10:1 to 200:1, or from 10:1 to 100:1, or from 10:1 to 50:1, or from 10:1 to 30:1, or from 10:1 to 20:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to therapeutically active agent in the composition is at least 20:1 (absorption enhancer: therapeutically active agent), optionally in a range of from 20:1 to 1000:1, or from 20:1 to 500:1, or from 20:1 to 300:1, or from 20:1 to 200:1, or from 20:1 to 100:1, or from 20:1 to 50:1, or from 20:1 to 30:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to therapeutically active agent in the composition is at least 30:1 (absorption enhancer: therapeutically active agent), optionally in a range of from 30:1 to 1000:1, or from 30:1 to 500:1, or from 30:1 to 300:1, or from 30:1 to 200:1, or from 30:1 to 100:1, or from 30:1 to 50:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzo yl)aminocaprylate).

In some embodiments of any one of the embodiments described herein, a weight ratio of the absorption enhancer to therapeutically active agent in the composition is at least 50:1 (absorption enhancer: therapeutically active agent), optionally in a range of from 50:1 to 1000:1, or from 50:1 to 500:1, or from 50:1 to 300:1, or from 50:1 to 200:1, or from 50:1 to 100:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to therapeutically active agent in the composition is at least 100:1 (absorption enhancer: therapeutically active agent), optionally in a range of from 100:1 to 1000:1, or from 100:1 to 500:1, or from 100:1 to 300:1, or from 100:1 to 200:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to therapeutically active agent in the composition is at least 200:1 (absorption enhancer: therapeutically active agent), optionally in a range of from 200:1 to 1000:1, or from 200:1 to 500:1, or from 200:1 to 300:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to therapeutically active agent in the composition is at least 300:1 (absorption enhancer: therapeutically active agent), optionally in a range of from 300:1 to 1000:1, or from 300:1 to 500:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to therapeutically active agent in the composition is at least 500:1,optionally in a range of from 500:1 to 1000:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

### Uses and Indications

In some embodiments of any of the embodiments described herein, the composition according to any of the respective embodiments described herein is for use in the treatment of a condition treatable by the therapeutically active agent (according to any of the respective embodiments described herein), the treatment comprising oral administration of the composition.

According to an aspect of some embodiments of the invention, there is provided a method of treating a condition treatable by oral administration of a therapeutically active agent (according to any of the respective embodiments described herein) in a subject in need thereof, the method comprising orally administering to the subject a composition according to any of the respective embodiments described herein comprising the respective therapeutically active agent (according to any of the respective embodiments described herein).

Condition treatable by the therapeutically active agent include conditions in which activity of GLP-2 and/or analogs thereof (e.g., GLP-2 receptor agonists) is beneficial.

Examples of conditions treatable according to embodiments of the invention include, without limitation, chemotherapy-induced adverse effects (e.g., abdominal cramping, bacterial translocation, diarrhea, gastrointestinal bleeding, malabsorption, mucositis and/or vomiting), cul-de-sac syndrome, digestion disorders, enteritis (e.g., radiation enteritis and/or infectious or post-infectious enteritis), inflammatory bowel disease, malabsorption syndromes, malnutrition (e.g., cachexia and/or anorexia), short-bowel syndrome (e.g., associated with surgical resection, congenital defect and/or disease-associated loss of absorption in the bowel), small intestine damage (e.g., associated with chemotherapy and/or toxic agents), celiac sprue (e.g., arising from gluten-induced enteropathy or celiac disease), tropical sprue, hypogammaglobulinemic sprue, ulcers (e.g., peptic ulcers, drug-induced ulcers, and/or ulcers associated with a pathogen), and ulcerative colitis.

Without being bound by any particular theory, it is believed that compositions described herein facilitate absorption in the stomach (rather than in the intestines), and that this is particularly advantageous in treating conditions in which intestinal function may be deficient, such as short bowel syndrome.

For conditions associated with chemotherapy, according to any of the respective embodiments described herein, the chemotherapeutic agent may be, for example, altretamine, bleomycin, busulfan, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gemcitabine, hydroxycarbamide, idarubicin, ifosfamide, irinotecan, liposomal doxorubicin, leucovorin, lomustine, melphalan, mercaptopurine, mesna, methotrexate, mitomycin, mitoxantrone, oxaliplatin, paclitaxel, pemetrexed, pentostatin, procarbazine, raltitrexed, streptozocin, tegafur-uracil, temozolomide, thiotepa, thioguanine, topotecan, treosulfan, vinblastine, vincristine, vindesine, and/or vinorelbine.

Therapeutically active agents (e.g., GLP-2 homologs) according to embodiments of the invention may optionally exhibit one or more activity in addition to GLP-2 activity (GLP-2 receptor agonist activity); for example, optionally being a GLP-1 receptor/GLP-2 receptor dual agonist, a glucagon receptor/GLP-2 receptor dual agonist, a gastric inhibitory polypeptide receptor (GIP)/GLP-2 receptor dual agonist, and/or a GLP-2 receptor/GLP-1 receptor/glucagon receptor triple agonist.

It is expected that during the life of a patent maturing from this application many relevant treatments of conditions by GLP-2 and GLP-2 receptor agonists will be developed, and the scope of the phrases “therapeutically active agent” and “condition treatable by . . . therapeutically active agent” are intended to include all such new technologies a priori.

### Formulation of Compositions

Each of the compositions and unit dosage forms described herein optionally consist essentially of the functional ingredients described hereinabove (e.g., a therapeutically active agent, an absorption enhancer, and an alkaline group-containing polymer, according to any of the embodiments described herein in any of the respective sections herein), or alternatively, the composition further comprises suitable pharmaceutically acceptable carriers and/or excipients.

Hereinafter, the phrases “physiologically acceptable carrier” and “pharmaceutically acceptable carrier”, which may be interchangeably used, refer to a carrier or a diluent that does not cause significant irritation to an organism and does not abrogate the activity (e.g., biological activity) and properties of the functional ingredient (e.g., a therapeutically active agent). An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch (e.g., types of non-modified starch), cellulose derivatives, gelatin, vegetable oils and polyethylene glycols.

In some embodiments of any one of the embodiments described herein, the composition is formulated as a solid composition, e.g., a solid unit dosage form. In some embodiments, the composition is formulated as a tablet or as a combination of tablets (e.g., a plurality of minitablets). In some embodiments, the composition is formulated as a capsule, as described herein, which comprises, for example, a plurality of minitablets and/or a powder, and which has a coating that is dissolvable in a gastric fluid (e.g., in gastric pH) or otherwise releases the capsule's content immediately (e.g., within less than 5 minutes, or less tghan 2 minutes or less than 1 minutes) upon contacting a gastric fluid.

Ingredients of the formulation (including, but not limited to, any two or more of the alkaline group-containing polymer, absorption enhancer and therapeutically active agent of any of the respective embodiments described herein) may optionally be mixed in a homogeneous manner or be distributed throughout the composition in a heterogeneous manner.

Techniques for formulation and administration of drugs may be found in “Remington's Pharmaceutical Sciences,” Mack Publishing Co., Easton, PA, latest edition, which is incorporated herein by reference.

Pharmaceutical compositions and unit dosage forms of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions and unit dosage forms for use in accordance with some embodiments of the invention may thus be formulated in conventional manner using one or more physiologically acceptable carriers comprising excipients and auxiliaries, which facilitate processing of the active ingredients into preparations which, can be used pharmaceutically.

The pharmaceutical composition and unit dosage forms can be formulated readily by combining the active compounds with pharmaceutically acceptable carriers well known in the art as being suitable for oral administration. Such carriers optionally facilitate formulation of the pharmaceutical composition as tablets (including minitablets), pellets, pills, dragees, capsules, powders, granules, elixirs, tinctures, liquids, gels, syrups, slurries, suspensions, emulsions and the like, for oral administration to a patient. Pharmacological preparations for oral use can be made using a solid excipient, optionally grinding the resulting mixture, and processing the mixture of granules, after adding suitable auxiliaries if desired, to obtain tablets or dragee cores. Pharmaceutical composition and unit dosage forms suitable for oral administration include, but are not limited to, rapid-release, time controlled-release, extended-release, and delayed-release pharmaceutical dosage forms. In some of any of the respective embodiments, a pharmaceutical composition formulated for oral administration is a solid composition, for example, tablet, capsule, powder or granules.

Suitable excipients are, in particular, fillers such as sugars, including lactose, sucrose, mannitol, or sorbitol; cellulose preparations such as, for example, methyl cellulose and/or hydroxypropylmethyl-cellulose; non-modified starches such as maize, wheat, rice and/or potato starch; gelatin; gum tragacanth; and/or physiologically acceptable polymers such as polyvinylpyrrolidone (PVP). If desired, lubricants may be added, such as talc or magnesium stearate.

In some embodiments of any one of the embodiments described herein, any one of the compositions or unit dosage forms described herein (e.g., formulated as a tablet) further comprises a lubricant. In some embodiments, the lubricant is included in a concentration of 5 weight percent or less, optionally 2 weight percent or less, and optionally about 1 weight percent. In some embodiments, the composition or unit dosage form described herein (e.g., formulated as a tablet) consists essentially of the therapeutically active agent (as described herein), absorption enhancer, lubricant, alkaline group-containing polymer, and optional protease inhibitor (as described herein). In some embodiments, the lubricant is magnesium stearate.

The compositions or unit dosage forms described herein (e.g., formulated as a tablet) may alternatively or in addition comprise additives and/or additional agents, such as, for example, antioxidants, solvents, odor absorbers, chelating agents, preservatives, thickeners, colorants or coloring agents (pigments, nacres, water-soluble dyestuffs), dispersants, fillers, flavoring agents and bactericides.

Dragee cores are optionally provided with suitable coatings. For this purpose, concentrated sugar solutions may be used which may optionally contain gum arabic, talc, polyvinylpyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin as well as soft, sealed capsules made of gelatin and a plasticizer, such as glycerol or sorbitol, pullulan, or HPMC. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added.

In some of any of the embodiments described herein, the pharmaceutical composition and/or unit dosage form according to any of the respective embodiments described herein is devoid of a gastroenteric coating, or otherwise comprises a coating that is dissolvable in the GI tract (e.g., in gastric fluid) and/or which otherwise releases the contents immediately (e.g., within no more than 5 minutes, or no more than 2 minutes or no more than one minute) upon contacting a gastric fluid.

Pharmaceutical compositions suitable for use in context of some embodiments of the invention include compositions wherein the therapeutically active agent is contained in an amount effective to achieve the intended purpose. More specifically, the composition preferably comprises a therapeutically effective amount of therapeutically active agent, that is, an amount of therapeutically active agent effective to prevent, alleviate or ameliorate symptoms of a disorder or prolong the survival of the subject being treated. Furthermore, an amount of absorption enhancer is preferably effective for enhancing absorption of the therapeutically active agent (e.g., in a manner described herein); and an amount of protease inhibitor (if present) is preferably effective for inhibiting degradation of the therapeutically active agent (e.g., a polypeptide agent) by a protease.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the therapeutically active agent described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation and dosage can be chosen by the individual physician in view of the patient's condition. (See e.g., Fingl et al., 1975, in “The Pharmacological Basis of Therapeutics”, Ch. 1 p.1).

Dosage amount and interval may be adjusted individually to provide levels (e.g., plasma levels) of the therapeutically active agent sufficient to induce or suppress a biological effect (minimal effective concentration, MEC). The MEC will vary for each preparation, but can be estimated from in vitro data. Dosages necessary to achieve the MEC will depend on individual characteristics. Detection assays can be used to determine plasma concentrations.

In some of any of the embodiments described herein, a dosage of the therapeutically active agent according to any of the respective embodiments described herein (e.g., an amount of the active agent in a unit dosage form composition, according to any of the respective embodiments described herein) is at least 1 mg of therapeutically active agent. In some such embodiments, the dosage is at least 3 mg of therapeutically active agent. In such embodiments, the dosage is at least 10 mg of therapeutically active agent. In such embodiments, the dosage is at least 30 mg of therapeutically active agent. In such embodiments, the dosage is at least 100 mg of therapeutically active agent. In such embodiments, the dosage is at least 300 mg of therapeutically active agent. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some of any of the embodiments described herein, a dosage of the therapeutically active agent according to any of the respective embodiments described herein (e.g., an amount of the active agent in a unit dosage form composition, according to any of the respective embodiments described herein) is 1,000 mg or less of therapeutically active agent (e.g., from 1 to 1,000 mg, or from 3 to 1,000 mg, or from 10 to 1,000 mg, or from 30 to 1,000 mg, or from 100 to 1,000 mg, or from 300 to 1,000 mg). In some such embodiments, the dosage is 500 mg or less of therapeutically active agent (e.g., from 1 to 500 mg, or from 3 to 500 mg, or from 10 to 50 mg, or from 30 to 500 mg, or from 100 to 500 mg, or from 300 to 500 mg). In some embodiments, the dosage is 300 mg or less of therapeutically active agent (e.g., from 1 to 3,000 ug, or from 3 to 300 mg, or from 10 to 300 mg, or from 30 to 300 mg, or from 100 to 300 mg). In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

Depending on the severity and responsiveness of the condition to be treated, dosing can be of a single or a plurality of administrations, with course of treatment lasting from several hours to several weeks or until cure is effected or diminution of the disease state is achieved.

The amount of a composition to be administered will, of course, be dependent on the subject being treated, the severity of the affliction, the manner of administration, the judgment of the prescribing physician, etc.

Each dosing may optionally be effected using a single dosage form, or a plurality of unit dosage forms. A plurality of unit dosage forms may optionally be used merely for convenience (e.g., wherein two or three unit dosage forms are used), or alternatively, to reduce variability in absorption according to any of the respective embodiments described in International Patent Application Publication No. WO 2018/033927, for example, wherein at least three or at least four unit dosage forms are used (e.g., from 3 to 10 or from 4 to 10, including any intermediate values and subranges therebetween).

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention may also be prepared (e.g., as described herein), placed in an appropriate container, and labeled for treatment of an indicated condition, as is further detailed herein.

In some embodiments of any one of the embodiments described herein, when the therapeutically active agents is a polypeptide, the composition further comprises at least one protease inhibitor, for example, a type of protease inhibitor, a concentration and/or absolute amount of protease inhibitor, and/or a ratio of protease inhibitor to absorption enhancer and/or therapeutically active agent according to any one of the embodiments relating to a protease inhibitor described in any of U.S. Patent Application Publication No. 2011/0142800, and

International Patent Application Publications WO 2016/128972 and WO 2018/033927, the contents of each of which (especially contents relating to protease inhibitors) are incorporated herein.

Herein throughout, the term “protease inhibitor” refers to a compound which reduces a proteolytic activity of a protease, for example, a proteolytic activity which inactivates a therapeutically active agent described herein. The term “protease inhibitor” encompasses, for example, both large molecules (e.g., proteins) and small molecules, as well as both naturally occurring compounds and synthetic compounds.

In some embodiments of any of the embodiments described herein, the at least one protease inhibitor comprises at least one trypsin inhibitor. In some embodiments, the at least one protease inhibitor consists essentially of one or more trypsin inhibitor(s).

Examples of trypsin inhibitors which may be utilized in any one of the embodiments described herein include, without limitation, lima bean trypsin inhibitor, aprotinin, soybean trypsin inhibitor, ovomucoid trypsin inhibitor and any combination thereof. In some embodiments, the at least one trypsin inhibitor comprises soybean trypsin inhibitor (SBTI). In some embodiments, the at least one trypsin inhibitor (an optionally the at least one protease inhibitor) consists essentially of SBTI.

In some embodiments of any of the embodiments described herein, the at least one protease inhibitor comprises at least one serpin. In some embodiments, the at least one protease inhibitor consists essentially of one or more serpin(s).

Examples of serpins which may be utilized in any one of the embodiments described herein, include, without limitation, alpha 1-antitrypsin, antitrypsin-related protein, alpha 1-antichymotrypsin, kallistatin, protein C inhibitor, cortisol binding globulin, thyroxine-binding globulin, angiotensinogen, centerin, protein Z-related protease inhibitor, vaspin, monocyte/neutrophil elastase inhibitor, plasminogen activator inhibitor-2, squamous cell carcinoma antigen-1 (SCCA-1), squamous cell carcinoma antigen-2 (SCCA-2), maspin, proteinase inhibitor 6 (PI-6), megsin, serpin B8 (PI-8), serpin B9 (PI-9), bomapin, yukopin, hurpin/headpin, antithrombin, heparin cofactor II, plasminogen activator inhibitor 1, glia-derived nexin, pigment epithelium derived factor, alpha 2-antiplasmin, complement 1-inhibitor, 47 kDa heat shock protein (HSP47), neuroserpin and pancpin.

In some embodiments of any of the embodiments described herein, the at least one protease inhibitor comprises at least one cysteine protease inhibitor. In some embodiments, the at least one protease inhibitor consists essentially of one or more cysteine protease inhibitor(s).

Examples of cysteine protease inhibitors which may be utilized in any one of the embodiments described herein include, without limitation, type 1 cystatins, type 2 cystatins, human cystatins C, D, S, SN, and SA, cystatin E/M, cystatin F, and type 3 cystatins (including kininogens).

In some embodiments of any of the embodiments described herein, the at least one protease inhibitor comprises at least one threonine protease inhibitor. In some embodiments, the at least one protease inhibitor consists essentially of one or more threonine protease inhibitor(s).

Examples of threonine protease inhibitors which may be utilized in any one of the embodiments described herein include, without limitation, bortezomib, MLN-519, ER-807446 and TMC-95A.

In some embodiments of any of the embodiments described herein, the at least one protease inhibitor comprises at least one aspartic protease inhibitor. In some embodiments, the at least one protease inhibitor consists essentially of one or more aspartic protease inhibitor(s).

Examples of aspartic protease inhibitors which may be utilized in any one of the embodiments described herein, include, without limitation, α₂-macroglobulin, pepstatin A, aspartic protease inhibitor 11, aspartic protease inhibitor 1, aspartic protease inhibitor 2, aspartic protease inhibitor 3, aspartic protease inhibitor 4, aspartic protease inhibitor 5, aspartic protease inhibitor 6, aspartic protease inhibitor 7, aspartic protease inhibitor 8, aspartic protease inhibitor 9, pepsin inhibitor Dit33, and protease A inhibitor 3.

In some embodiments of any of the embodiments described herein, the at least one protease inhibitor comprises at least one metalloprotease inhibitor. In some embodiments, the at least one protease inhibitor consists essentially of one or more metalloprotease inhibitor(s).

Examples of metalloprotease inhibitors which may be utilized in any one of the embodiments described herein, include, without limitation, angiotensin-1-converting enzyme inhibitory peptide, antihemorrhagic factor BJ46a, beta-casein, proteinase inhibitor CeKI, venom metalloproteinase inhibitor DM43, carboxypeptidase A inhibitor, smpI, IMPI, alkaline proteinase, latexin, carboxypeptidase inhibitor, antihemorrhagic factor HSF, testican-3, SPOCK3, TIMP1, metalloproteinase inhibitor 1, metalloproteinase inhibitor 2, TIMP2, metalloproteinase inhibitor 3, TIMP3, metalloproteinase inhibitor 4, TIMP4, putative metalloproteinase inhibitor tag-225, tissue inhibitor of metalloprotease, WAP, kazal inhibitor, immunoglobulin, and kunitz and NTR domain-containing protein 1.

Examples of protease inhibitors which may be utilized in any one of the embodiments described herein also include, without limitation, AEBSF-HCl, ε-aminocaproic acid, α1-antichymotypsin, antipain, antithrombin III, α1-antitrypsin, APMSF (4-amidinophenyl-methane sulfonyl-fluoride), sprotinin, benzamidine, chymostatin, DFP (diisopropylfluoro-phosphate), leupeptin, 4-(2-Aminoethyl)-benzenesulfonyl fluoride hydrochloride, PMSF (phenylmethyl sulfonyl fluoride), TLCK (1-chloro-3-tosylamido-7-amino-2-heptanone), TPCK (1-chloro-3-tosylamido-4-phenyl-2-butanone), pentamidine isothionate, pepstatin, guanidium, α2-macroglobulin, a chelating agent of zinc, and iodoacetate.

In some embodiments of any one of the embodiments described herein, the amount of a protease inhibitor in a unit dosage form described herein is at least about 0.1 mg. In some embodiments, the amount of a protease inhibitor in a unit dosage form described herein is at least about 0.2 mg. In some embodiments, the amount of a protease inhibitor in a unit dosage form described herein is at least about 0.3 mg. In some embodiments, the amount of a protease inhibitor in a unit dosage form described herein is at least about 0.4 mg. In some embodiments, the amount of a protease inhibitor in a unit dosage form described herein is at least about 0.6 mg. In some embodiments, the amount of a protease inhibitor in a unit dosage form described herein is at least about 0.8 mg. In some embodiments, the amount of a protease inhibitor in a unit dosage form described herein is at least about 1 mg. In some embodiments, the amount of a protease inhibitor in a unit dosage form described herein is at least about 1.5 mg. In some embodiments, the amount of a protease inhibitor in a unit dosage form described herein is at least about 2 mg. In some embodiments, the amount of a protease inhibitor in a unit dosage form described herein is at least about 2.5 mg. In some embodiments, the amount of a protease inhibitor in a unit dosage form described herein is at least about 3 mg. In some embodiments, the amount of a protease inhibitor in a unit dosage form described herein is at least about 5 mg. In some embodiments, the amount of a protease inhibitor in a unit dosage form described herein is at least about 7 mg. In some embodiments, the amount of a protease inhibitor in a unit dosage form described herein is at least about 10 mg. In some embodiments, the amount of a protease inhibitor in a unit dosage form described herein is at least about 12 mg. In some embodiments, the amount of a protease inhibitor in a unit dosage form described herein is at least about 15 mg. In some embodiments, the amount of a protease inhibitor in a unit dosage form described herein is at least about 20 mg. In some embodiments, the amount of a protease inhibitor in a unit dosage form described herein is at least about 30 mg. In some embodiments, the amount of a protease inhibitor in a unit dosage form described herein is at least about 50 mg. In some embodiments, the amount of a protease inhibitor in a unit dosage form described herein is at least about 70 mg. In some embodiments, the amount of a protease inhibitor in a unit dosage form described herein is at least about 100 mg.

In some embodiments of any one of the embodiments described herein, the amount of a protease inhibitor in a unit dosage form described herein is in a range of from 0.1 to 1 mg. In some embodiments, the amount of a protease inhibitor in a unit dosage form described herein is in a range of from 0.2 to 1 mg. In some embodiments, the amount of a protease inhibitor in a unit dosage form described herein is in a range of from 0.3 to 1 mg. In some embodiments, the amount of a protease inhibitor in a unit dosage form described herein is in a range of from 0.5 to 1 mg.

In some embodiments of any one of the embodiments described herein, the amount of a protease inhibitor in a unit dosage form described herein is in a range of from 0.1 to 2 mg. In some embodiments, the amount of a protease inhibitor in a unit dosage form described herein is in a range of from 0.2 to 2 mg. In some embodiments, the amount of a protease inhibitor in a unit dosage form described herein is in a range of from 0.3 to 2 mg. In some embodiments, the amount of a protease inhibitor in a unit dosage form described herein is in a range of from 0.5 to 2 mg. In some embodiments, the amount of a protease inhibitor in a unit dosage form described herein is in a range of from 1 to 2 mg.

In some embodiments of any one of the embodiments described herein, the amount of a protease inhibitor in a unit dosage form described herein is in a range of from 1 to 10 mg. In some embodiments, the amount of a protease inhibitor in a unit dosage form described herein is in a range of from 2 to 10 mg. In some embodiments, the amount of a protease inhibitor in a unit dosage form described herein is in a range of from 3 to 10 mg. In some embodiments, the amount of a protease inhibitor in a unit dosage form described herein is in a range of from 5 to 10 mg.

In some embodiments of any one of the embodiments described herein, the amount of a protease inhibitor in a unit dosage form described herein is in a range of from 1 to 20 mg. In some embodiments, the amount of a protease inhibitor in a unit dosage form described herein is in a range of from 2 to 20 mg. In some embodiments, the amount of a protease inhibitor in a unit dosage form described herein is in a range of from 3 to 20 mg. In some embodiments, the amount of a protease inhibitor in a unit dosage form described herein is in a range of from 5 to 20 mg. In some embodiments, the amount of a protease inhibitor in a unit dosage form described herein is in a range of from 10 to 20 mg.

In some embodiments of any one of the embodiments described herein, the amount of a protease inhibitor in a unit dosage form described herein is in a range of from 10 to 100 mg. In some embodiments, the amount of a protease inhibitor in a unit dosage form described herein is in a range of from 20 to 100 mg. In some embodiments, the amount of a protease inhibitor in a unit dosage form described herein is in a range of from 30 to 100 mg. In some embodiments, the amount of a protease inhibitor in a unit dosage form described herein is in a range of from 50 to 100 mg.

In some embodiments of any one of the embodiments described herein, the amount of a protease inhibitor in a unit dosage form described herein is in a range of from 10 to 200 mg. In some embodiments, the amount of a protease inhibitor in a unit dosage form described herein is in a range of from 20 to 200 mg. In some embodiments, the amount of a protease inhibitor in a unit dosage form described herein is in a range of from 30 to 200 mg. In some embodiments, the amount of a protease inhibitor in a unit dosage form described herein is in a range of from 50 to 200 mg. In some embodiments, the amount of a protease inhibitor in a unit dosage form described herein is in a range of from 100 to 200 mg.

In some embodiments of any one of the embodiments described herein, the amount of a protease inhibitor in a unit dosage form described herein is at least about 10 kallikrein inactivator units (k.i.u.). In some embodiments, the amount of a protease inhibitor in a unit dosage form described herein is at least about 12 k.i.u. In some embodiments, the amount of a protease inhibitor in a unit dosage form described herein is at least about 15 k.i.u. In some embodiments, the amount of a protease inhibitor in a unit dosage form described herein is at least about 20 k.i.u. In some embodiments, the amount of a protease inhibitor in a unit dosage form described herein is at least about 30 k.i.u. In some embodiments, the amount of a protease inhibitor in a unit dosage form described herein is at least about 40 k.i.u. In some embodiments, the amount of a protease inhibitor in a unit dosage form described herein is at least about 50 k.i.u. In some embodiments, the amount of a protease inhibitor in a unit dosage form described herein is at least about 70 k.i.u. In some embodiments, the amount of a protease inhibitor in a unit dosage form described herein is at least about 100 k.i.u. In some embodiments, the amount of a protease inhibitor in a unit dosage form described herein is at least about 150 k.i.u. In some embodiments, the amount of a protease inhibitor in a unit dosage form described herein is at least about 200 k.i.u. In some embodiments, the amount of a protease inhibitor in a unit dosage form described herein is at least about 300 k.i.u. In some embodiments, the amount of a protease inhibitor in a unit dosage form described herein is at least about 500 k.i.u. In some embodiments, the amount of a protease inhibitor in a unit dosage form described herein is at least about 700 k.i.u. In some embodiments, the amount of a protease inhibitor in a unit dosage form described herein is at least about 1000 k.i.u. In some embodiments, the amount of a protease inhibitor in a unit dosage form described herein is at least about 1500 k.i.u. In some embodiments, the amount of a protease inhibitor in a unit dosage form described herein is at least about 3000 k.i.u. In some embodiments, the amount of a protease inhibitor in a unit dosage form described herein is at least about 4000 k.i.u. In some embodiments, the amount of a protease inhibitor in a unit dosage form described herein is at least about 5000 k.i.u.

Herein and in the art, a “kallikrein inactivating unit” (k.i.u) refers to an amount of protease inhibitor that has the ability to inhibit 2 units of kallikrein by 50% (e.g., in aqueous solution at an optimal pH and solution volume for activity of the protease inhibitor).

In some embodiments of any one of the embodiments described herein, a weight ratio of protease inhibitor to therapeutically active agent is in a range of from 1:1 to 5:1 (protease inhibitor: therapeutically active agent). In some embodiments, a weight ratio of protease inhibitor to therapeutically active agent is in a range of from 5:1 to 10:1. In some embodiments, a weight ratio of protease inhibitor to therapeutically active agent is in a range of from 10:1 to 20:1. In some embodiments, a weight ratio of protease inhibitor to therapeutically active agent is in a range of from 20:1 to 30:1. In some embodiments, a weight ratio of protease inhibitor to therapeutically active agent is in a range of from 30:1 to 40:1. In some embodiments, a weight ratio of protease inhibitor to therapeutically active agent is in a range of from 40:1 to 50:1. In some embodiments, a weight ratio of protease inhibitor to therapeutically active agent is in a range of from 50:1 to 75:1. In some embodiments, a weight ratio of protease inhibitor to therapeutically active agent is in a range of from 75:1 to 100:1. In some embodiments, a weight ratio of protease inhibitor to therapeutically active agent is in a range of from 100:1 to 200:1. In some embodiments, a weight ratio of protease inhibitor to therapeutically active agent is in a range of from 200:1 to 300:1. In some embodiments, a weight ratio of protease inhibitor to therapeutically active agent is in a range of from 300:1 to 400:1. In some embodiments, a weight ratio of protease inhibitor to therapeutically active agent is in a range of from 400:1 to 500:1. In some embodiments, the protease inhibitor is soybean trypsin inhibitor.

### Additional Definitions

Herein, the term “polypeptide” refers to a polymer comprising at least 4 amino acid residues linked by peptide bonds or analogs thereof (as described herein), and optionally only by peptide bonds per se. In some embodiments, the polypeptide comprises at least 10 amino acid residues or analogs thereof. In some embodiments, the polypeptide comprises at least 20 amino acid residues or analogs thereof. In some embodiments, the polypeptide comprises at least 30 amino acid residues or analogs thereof. In some embodiments, the polypeptide comprises at least 50 amino acid residues or analogs thereof. The term “polypeptide” encompasses native polypeptides (e.g., degradation products, synthetically synthesized polypeptides and/or recombinant polypeptides), including, without limitation, native proteins, fragments and substituted derivatives of native proteins and homologs of native proteins and/or fragments and/or substituted derivatives thereof; as well as peptidomimetics (typically, synthetically synthesized polypeptides) and peptoids and semipeptoids which are polypeptide analogs, which may have, for example, modifications rendering the polypeptides more stable while in a body or more capable of penetrating into cells. Such modifications include, but are not limited to N-terminus modification (e.g., by an acyl group), C-terminus modification (e.g., via amidation, as in apraglutide and glepaglutide), peptide bond modification, backbone modifications, and residue modification. Methods for preparing peptidomimetic compounds are well known in the art and are specified, for example, in Quantitative Drug Design, C.A. Ramsden Gd., Chapter 17.2, F. Choplin Pergamon Press (1992), which is incorporated by reference as if fully set forth herein. Further details in this respect are provided herein.

Peptide bonds (—CO—NH—) within the polypeptide may be substituted, for example, by N-methylated amide bonds (—N(CH₃)—CO—), ester bonds (—C(═O)—O—), ketomethylene bonds (—CO—CH₂—), sulfinylmethylene bonds (—S(═O)—CH₂—), α-aza bonds (—NH—N(R)—CO—), wherein R is any alkyl (e.g., methyl), amine bonds (—CH₂—NH—), sulfide bonds (—CH₂—S—), ethylene bonds (—CH₂—CH₂—), hydroxyethylene bonds (—CH(OH)—CH₂—), thioamide bonds (—CS—NH—), olefinic double bonds (—CH═CH—), fluorinated olefinic double bonds (—CF═CH—), retro amide bonds (—NH—CO—), peptide derivatives (—N(R)—CH2—CO—), wherein R is the “normal” side chain, naturally present on the carbon atom.

These modifications can occur at any of the bonds along the polypeptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The polypeptides of some embodiments of the invention (e.g., a therapeutically active agent described herein) may also include one or more modified amino acids or one or more non-amino acid monomers (e.g. fatty acids, complex carbohydrates etc.).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables A and B below list naturally occurring amino acids (Table A), and non-conventional or modified amino acids (e.g., synthetic, Table B) which can be used with some embodiments of the invention.

TABLE A

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE B

| Non-conventional amino acid | Code | Non-conventional amino acid | Code |
|---|---|---|---|
| ornithine | Orn | hydroxyproline | Hyp |
| α-aminobutyric acid | Abu | aminonorbornyl-carboxylate | Norb |
| D-alanine | Dala | aminocyclopropane-carboxylate | Cpro |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl) glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl-homophenylalanine | Nmhphe |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentyl-alanine | Cpen | N-methyl-cyclopentylalanine | Nmcpen |
| α-amino-α-methylbutyrate | Aabu | N-methyl-α-amino-α-methylbutyrate | Nmaabu |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl-homophenylalanine | Mhphe |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2-diphenylethyl)glycine | Nbhm | N-(N-(2,2-diphenylethyl) carbamylmethyl-glycine | Nnbhm |
| N-(3,3-diphenylpropyl)glycine | Nbhe | N-(N-(3,3-diphenylpropyl) carbamylmethyl-glycine | Nnbhe |
| 1-carboxy-1-(2,2-diphenyl ethylamino)cyclopropane | Nmbc | 1,2,3,4-tetrahydroisoquinoline-3-carboxylic acid | Tic |
| phosphoserine | pSer | phosphothreonine | pThr |
| phosphotyrosine | pTyr | O-methyl-tyrosine | |
| 2-aminoadipic acid | | hydroxylysine | |

The polypeptides of some embodiments of the invention (e.g., a therapeutically active agent described herein) are preferably utilized in a linear form, although it will be appreciated that in cases where cyclization does not severely interfere with polypeptide characteristics, cyclic forms of the polypeptide can also be utilized.

In some embodiments of any one of the embodiments described herein, the polypeptide is water-soluble, as defined herein.

Water-soluble polypeptides preferably include one or more non-natural or natural polar amino acids, including but not limited to serine and threonine which are capable of increasing polypeptide water-solubility due to their hydroxyl-containing side chain. Optionally, a homolog of a polypeptide is selected so as to be more water-soluble than the parent polypeptide, for example, by replacing one or more amino acids in the polypeptide with polar amino acids.

The polypeptides of some embodiments of the invention (e.g., a therapeutically active agent described herein) may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing polypeptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final polypeptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the polypeptide compounds of some embodiments of the invention (e.g., a therapeutically active agent described herein) involves solid phase peptide synthesis.

Large scale polypeptide synthesis is described by Andersson et al. [Biopolymers 2000; 55:227-250].

Herein, a “homolog” of a given polypeptide refers to a polypeptide that exhibits at least 80% homology, preferably at least 90% homology, and more preferably at least 95% homology, and more preferably at least 98% homology to the given polypeptide. In some embodiments, a homolog of a given polypeptide further shares a therapeutic activity with the given polypeptide. The percentage of homology refers to the percentage of amino acid residues in a first polypeptide sequence which match a corresponding residue of a second polypeptide sequence to which the first polypeptide is being compared. Generally, the polypeptides are aligned to give maximum homology. A variety of strategies are known in the art for performing comparisons of amino acid sequences in order to assess degrees of identity, including, for example, manual alignment, computer assisted sequence alignment and combinations thereof. A number of algorithms (which are generally computer implemented) for performing sequence alignment are widely available, or can be produced by one of skill in the art. Representative algorithms include, e.g., the local homology algorithm of Smith and Waterman (Adv. Appl. Math., 1981, 2: 482); the homology alignment algorithm of Needleman and Wunsch (J. Mol. Biol., 1970, 48: 443); the search for similarity method of Pearson and Lipman (Proc. Natl. Acad. Sci. (USA), 1988, 85: 2444); and/or by computerized implementations of these algorithms (e.g., GAP, BESTFIT, FASTA, and TFASTA in the Wisconsin Genetics Software Package Release 7.0, Genetics Computer Group, 575 Science Dr., Madison, Wis.). Readily available computer programs incorporating such algorithms include, for example, BLASTN, BLASTP, Gapped BLAST, PILEUP, CLUSTALW etc. When utilizing BLAST and Gapped BLAST programs, default parameters of the respective programs may be used. Alternatively, the practitioner may use non-default parameters depending on his or her experimental and/or other requirements (see for example, the Web site having URL www(dot)ncbi(dot)nlm(dot)nih(dot)gov).

Herein, the terms “amine” and “amino” each refer to a —NR′R″ group, wherein R′ and R″ are each hydrogen or a substituted or non-substituted alkyl, alkenyl, alkynyl, cycloalkyl, heteroalicyclic (linked to amine nitrogen via a ring carbon thereof), aryl, or heteroaryl (linked to amine nitrogen via a ring carbon thereof), as defined herein. Alternatively, R′ and R″ may optionally be linked to form a heteroalicyclic ring (as defined herein). Optionally, R′ and R″ and R″′ are hydrogen or alkyl comprising 1 to 4 carbon atoms. Optionally, at least one of R′ and R″ is hydrogen and optionally both are hydrogen. When substituted, the carbon atom of an R′ and R″ hydrocarbon moiety which is bound to the nitrogen atom of the amine is not substituted by oxo, such that R′ and R″ are not (for example) carbonyl, C-carboxy or amide, as these groups are defined herein.

As used herein throughout, the term “alkyl” refers to any saturated aliphatic hydrocarbon including straight chain and branched chain groups. Preferably, the alkyl group has 1 to 20 carbon atoms. Whenever a numerical range; e.g., “1 to 20”, is stated herein, it implies that the group, in this case the hydrocarbon, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or non-substituted. When substituted, the substituent group can be, for example, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

Herein, the term “alkenyl” describes an unsaturated aliphatic hydrocarbon comprise at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or non-substituted. Substituted alkenyl may have one or more substituents, whereby each substituent group can independently be, for example, alkynyl, cycloalkyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

Herein, the term “alkynyl” describes an unsaturated aliphatic hydrocarbon comprise at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or non-substituted. Substituted alkynyl may have one or more substituents, whereby each substituent group can independently be, for example, cycloalkyl, alkenyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

A “cycloalkyl” group refers to a saturated on unsaturated all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. When a cycloalkyl group is unsaturated, it may comprise at least one carbon-carbon double bond and/or at least one carbon-carbon triple bond.

An “aryl” group refers to an all-carbon monocyclic or fused-ring polycyclic (i.e., rings which share adjacent pairs of carbon atoms) having a completely conjugated pi-electron system. Examples, without limitation, of aryl groups are phenyl, naphthalenyl and anthracenyl. The aryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroaryl” group refers to a monocyclic or fused ring (i.e., rings which share an adjacent pair of atoms) having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or non-substituted. When substituted, the substituted group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. Representative examples are piperidine, piperazine, tetrahydrofuran, tetrahydropyran, morpholine and the like.

An “azide” group refers to a —N═N⁺=N⁻ group.

An “alkoxy” group refers to any of an —O-alkyl, —O-alkenyl, —O-alkynyl, —O-cycloalkyl, and —O-heteroalicyclic group, as defined herein.

An “aryloxy” group refers to both an —O-aryl and an —O-heteroaryl group, as defined herein.

A “hydroxy” group refers to a —OH group.

A “thiohydroxy” or “thiol” group refers to a —SH group.

A “thioalkoxy” group refers to any of an —S-alkyl, —S-alkenyl, —S-alkynyl, —S-cycloalkyl, and —S-heteroalicyclic group, as defined herein.

A “thioaryloxy” group refers to both an —S-aryl and an —S-heteroaryl group, as defined herein.

A “carbonyl” or “acyl” group refers to a —C(═O)—R′ group, where R′ is defined as hereinabove.

A “thiocarbonyl” group refers to a —C(═S)—R′ group, where R′ is as defined herein.

A “C-carboxy” group refers to a —C(═O)—O—R′ group, where R′ is as defined herein.

An “O-carboxy” group refers to an R′C(═O)—O— group, where R′ is as defined herein.

A “carboxylic acid” group refers to a —C(═O)OH group.

An “oxo” group refers to a ═O group.

An “imine” group refers to a ═N—R′ group, where R′ is as defined herein.

An “oxime” group refers to a ═N—OH group.

A “hydrazone” group refers to a ═N—NR′R″ group, where each of R′ and R″ is as defined herein.

A “halo” group refers to fluorine, chlorine, bromine or iodine.

A “sulfinyl” group refers to an —S(═O)—R′ group, where R′ is as defined herein.

A “sulfonyl” group refers to an —S(═O)₂—R′ group, where R′ is as defined herein.

A “sulfonate” group refers to an —S(═O)₂—O—R′ group, where R′ is as defined herein.

A “sulfate” group refers to an —O—S(═O)₂—O—R′ group, where R′ is as defined as herein.

A “sulfonamide” or “sulfonamido” group encompasses both S-sulfonamido and N-sulfonamido groups, as defined herein.

An “S-sulfonamido” group refers to a —S(═O)₂—NR′R″ group, with each of R′ and R″ as defined herein.

An “N-sulfonamido” group refers to an R′S(═O)₂—NR″— group, where each of R′ and R″ is as defined herein.

An “O-carbamyl” group refers to an —OC(═O)—NR′R″ group, where each of R′ and R″ is as defined herein.

An “N-carbamyl” group refers to an R′OC(═O)—NR″— group, where each of R′ and R″ is as defined herein.

An “O-thiocarbamyl” group refers to an —OC(═S)—NR′R″ group, where each of R′ and R″ is as defined herein.

An “N-thiocarbamyl” group refers to an R′OC(═S)NR″— group, where each of R′ and R″ is as defined herein.

An “S-thiocarbamyl” group refers to an —SC(═O)—NR′R″ group, where each of R′ and R″ is as defined herein.

An “amide” or “amido” group encompasses C-amido and N-amido groups, as defined herein.

A “C-amido” group refers to a —C(═O)—NR′R″ group, where each of R′ and R″ is as defined herein.

An “N-amido” group refers to an R′C(═O)—NR″— group, where each of R′ and R″ is as defined herein.

A “urea group” refers to an —N(R′)—C(═O)—NR″R″′ group, where each of R′, R″ and R″ is as defined herein.

A “thiourea group” refers to a —N(R′)—C(═S)—NR″R″′ group, where each of R′, R″ and R″ is as defined herein.

A “nitro” group refers to an —NO₂ group.

A “cyano” group refers to a —C≡N group.

The term “phosphonyl” or “phosphonate” describes a —P(═O)(OR′)(OR″) group, with R′ and R″ as defined hereinabove.

The term “phosphate” describes an —O—P(═O)(OR′)(OR″) group, with each of R′ and R″ as defined hereinabove.

The term “phosphinyl” describes a —PR′R″ group, with each of R′ and R″ as defined hereinabove.

The term “hydrazine” describes a —NR′—NR″R″′ group, with R′, R″, and R″′ as defined herein.

As used herein, the term “hydrazide” describes a —C(═O)—NR′—NR″R″′ group, where R′, R″ and R′″ are as defined herein.

As used herein, the term “thiohydrazide” describes a —C(═S)—NR′—NR″R″′ group, where R′, R″ and R′″ are as defined herein.

A “guanidinyl” group refers to an —RaNC(═NRd)—NRbRc group, where each of Ra, Rb, Rc and Rd can be as defined herein for R′ and R″.

A “guanyl” or “guanine” group refers to an RaRbNC(═NRd)— group, where Ra, Rb and Rd are as defined herein.

For any of the embodiments described herein, each of the compounds described herein (including therapeutically active agents, absorption enhancers and polymers) may be in a form of a salt, for example, a pharmaceutically acceptable salt, and/or in a form of a prodrug.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt and/or a base addition salt.

An acid addition salt comprises at least one basic (e.g., amine and/or guanidinyl) group of the compound which is in a positively charged form (e.g., wherein the basic group is protonated), in combination with at least one counter-ion, derived from the selected acid, that forms a pharmaceutically acceptable salt. The acid addition salts of the compounds described herein may therefore be complexes formed between one or more basic groups of the compound and one or more equivalents of an acid.

A base addition salt comprises at least one acidic (e.g., carboxylic acid) group of the compound which is in a negatively charged form (e.g., wherein the acidic group is deprotonated), in combination with at least one counter-ion, derived from the selected base, that forms a pharmaceutically acceptable salt. The base addition salts of the compounds described herein may therefore be complexes formed between one or more acidic groups of the compound and one or more equivalents of a base.

Depending on the stoichiometric proportions between the charged group(s) in the compound and the counter-ion in the salt, the acid additions salts and/or base addition salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof, and/or a carboxylate anion and a base addition salt thereof.

The base addition salts may include a cation counter-ion such as sodium, potassium, ammonium, calcium, magnesium and the like, that forms a pharmaceutically acceptable salt.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

As used herein, the term “prodrug” refers to a compound which is converted in the body to an active compound (e.g., the compound of the formula described hereinabove). A prodrug is typically designed to facilitate administration, e.g., by enhancing absorption. A prodrug may comprise, for example, the active compound modified with ester groups, for example, wherein any one or more of the hydroxyl groups of a compound is modified by an acyl group, optionally (C₁₋₄)-acyl (e.g., acetyl) group to form an ester group, and/or any one or more of the carboxylic acid groups of the compound is modified by an alkoxy or aryloxy group, optionally (C₁₋₄)-alkoxy (e.g., methyl, ethyl) group to form an ester group.

Further, each of the compounds described herein (including therapeutically active agents, absorption enhancers and polymers), including the salts thereof, can be in a form of a solvate or a hydrate thereof.

The term “solvate” refers to a complex of variable stoichiometry (e.g., di-, tri-, tetra-, penta-, hexa-, and so on), which is formed by a solute (the heterocyclic compounds described herein) and a solvent, whereby the solvent does not interfere with the biological activity of the solute.

The term “hydrate” refers to a solvate, as defined hereinabove, where the solvent is water.

The compounds described herein can be used as polymorphs and the present embodiments further encompass any isomorph of the compounds and any combination thereof.

The compounds and structures described herein encompass any stereoisomer, including enantiomers and diastereomers, of the compounds described herein, unless a particular stereoisomer is specifically indicated.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an (R) or an (S) configuration and any combination, and compounds according to some embodiments of the present invention, can have any their chiral centers exhibit an (R) or an (S) configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

### Materials

Croscarmellose sodium (Parteck® CCS; crosslinked; CAS No. 74811-65-7) was obtained from Merck.

Sodium alginate (CAS No. 9005-38-3), average MW 300-350 kDa, viscosity at room temperature 350-500 mPa (non-crosslinked), was obtained from Carl Roth.

Sodium carboxymethylcellulose (CMC), CAS No. 9004-32-4 (not cross linked), low viscosity (43 mPa at room temperature) was obtained from Merck or Ashland.

Sodium starch glycolate Type A (Primojel®; cross-linked; CAS No. 9063-38-1), average MW 2,000 kDa, viscosity at room temperature lower than 200 mPa, was obtained from DFE Pharma.

### pH Determination Using pH Meter

Test substances were dissolved in various volumes of 0.1 M HCl solution (pH 1.2) at concentrations of 1, 2, 4, 10, 20, 50, 100 and 150 mg/ml. The solution pH was determined using an MP-103 pH-meter (MRC, Israel) equipped with ELC-10-00 electrode (MRC, Israel) or with a thin electrode HI1083 (HANNA instruments Inc.), shortly after dissolution and up to 26 hours later in order to ensure that the pH remained stable. The highest pH value obtained during the experimental time period was recorded in order to compare the acid-neutralizing capacity of each substance.

Other pH measurements were performed by adding a suitable colorimetric pH indicator solution to the tested medium.

### Example 1

### Effect of Basic Polymers on pH in Simulated Gastric Fluid

The effects of basic polymers on gastric fluid and on the behavior of SNAC in gastric fluid was investigated using HCl solutions as simulated gastric fluid.

SNAC and the basic polymers sodium alginate, sodium carboxymethylcellulose in non-crosslinked (Na-CMC) and crosslinked (croscarmellose sodium, CCS) forms, and sodium starch glycolate (SSG) were dissolved in various volumes of 0.1 M HCl solution (pH 1.2) at concentrations of 1, 2, 4, 10, 20, 50, 100 and 150 mg/ml, and the solution pH was determined using an MP-103 pH-meter (MRC, Israel) equipped with ELC-10-00 electrode (MRC, Israel), as described in the Materials and Methods section hereinabove.

As shown in FIG. 1, upon addition to a HCl solution (pH 1.2), SNAC dissolved rapidly and elevated the pH in a concentration-dependent manner, wherein the pH 5 minutes after dissolution was about 1.5 upon addition of 10 mg/ml SNAC, 6.5 upon addition of 20 mg/ml SNAC, and about 7.5 upon addition of 100 mg/ml SNAC.

These results indicate that a substantial proportion of SNAC is converted under gastric conditions from a salt to the corresponding carboxylic acid (NAC), which is substantially less water soluble than the salt and which is not an effective permeation enhancer.

In order to obtain a beneficial rise in pH with a reduced loss of the active form of the absorption enhancer, the effect of various concentrations of different polymers containing carboxylate groups (sodium alginate, sodium starch glycolate, sodium carboxymethylcellulose, and croscarmellose sodium) on pH upon addition to a 0.1 M HCl solution (pH 1.2) was also evaluated. FIG. 2A presents pH values determined using an MP-103 pH-meter (MRC, Israel) equipped with ELC-10-00 electrode (MRC, Israel) and FIG. 2B presents pH values determined using an MP-103 pH-meter (MRC, Israel) equipped with a thin electrode HI1083 (HANNA instruments Inc.).

As shown in FIGS. 2A and 2B, each of the tested polymers exhibited a concentration-dependent increase in pH, with pH values of about 4-4.5 being obtained at the higher concentrations tested.

The sodium alginate, Na-CMC, CCS and sodium starch glycolate tested powders form gels (of various viscosities) upon addition to aqueous media, and the dispersion of the polymers in such gels may be inhomogeneous. In order to assess whether the acid-neutralizing capacity of the polymer is determined by the concentration of the polymer in gel, preliminary studies were performed with polymers in a powder form inserted to the acidic medium without mixing. pH values were determined using a suitable pH indicator solution.

The polymers in powder form produced local acid-neutralizing effects: sodium starch glycolate dissolved quickly with strong acid-neutralization, croscarmellose sodium dissolved quickly with weaker acid-neutralization, sodium carboxymethylcellulose did not dissolve well but exhibited some acid-neutralization, and sodium alginate exhibited little acid-neutralization (data not shown).

These results indicate that of the tested polymers, sodium starch glycolate (and to some extent, croscarmellose sodium) has a particularly strong local acid-neutralizing effect in powder form, rendering it particularly suitable for non-enterically coated formulations with an absorption enhancer such as SNAC.

Without being bound by any particular theory, it is believed that the limited effect of sodium carboxymethylcellulose and sodium alginate is associated with slow penetration of the water into the polymeric matrix, whereas the particularly effective acid neutralization of sodium starch glycolate is at least partially associated with its rapid water absorption (swelling).

As sodium starch glycolate and croscarmellose sodium exhibited the strongest local acid-neutralizing capacities, additional experiments were performed with these polymers.

To assess the maximal acid-neutralizing capacity of the polymers, experiments were repeated with each polymer compressed to a form of a tablet.

In preliminary studies, flat round 100 mg tablets were formed from sodium starch glycolate or croscarmellose sodium using a 6 mm punch and 2 ton compression force, and the tablets were placed into 40 ml HCl solutions (static dissolution) at various pH levels—1.2, 1.5 or 2.0. A temperature of 37° C. was maintained, using a water bath. The resulting pH values were determined using a suitable pH indicator solution.

Although sodium starch glycolate and croscarmellose sodium tablets exhibited different swelling patterns, they exhibited comparable neutralization strength in HCl solutions (data not shown). Sodium starch glycolate tablets resulted in stronger acid neutralization than did croscarmellose sodium tablets for each tested initial pH (wherein the efficacy of local acid neutralization was greater when the initial pH was higher).

These results indicate that although sodium starch glycolate and croscarmellose sodium may have comparable efficacy in increasing the pH of gastric fluid to at least about 4, sodium starch glycolate is more effective than croscarmellose sodium at raising the pH of gastric fluid to at least about 5, indicating that sodium starch glycolate is more effective at acid neutralization than is croscarmellose sodium.

In additional studies, 100 mg sodium starch glycolate tablets were prepared with an 8 mm round punch and 1.5 ton compression force. The tablets were dissolved without shaking in weighing boats containing 50 ml 0.01 M HCl solutions (pH 2.0, 37° C.), and local pH was determined.

Determination of pH in the proximity of the tablets, by adding an indicator to the solution, indicated that the local pH was about 7.

These results indicate that sodium starch glycolate increases local pH to well above pH 5 (the pKa of SNAC), thereby facilitating dissolution of SNAC at concentrations effective for the action of SNAC and related compounds as an absorption enhancer, as well as inhibiting peptide degradation by pepsin (which is active only at low pH).

Without being bound by any particular theory, it is believed that the local nature of the observed pH increase is associated with the viscous gel which forms upon polymer dissolution (hydration and relaxation upon wetting), which limits the transport of acid from outside to the local region with higher pH. It is further believed that due to this inhomogeneity, determination of local pH presents a more relevant parameter than pH of the medium as a whole. For example, the generally smaller increases in pH of the medium as a whole (FIGS. 3A-B) may be explained as reflecting an average of the pH of the region proximal to the tablets in which acid is neutralized effectively and the pH of distal regions in which pH is less affected.

In additional studies, 100 mg tablets were formed from various polymers (e.g., sodium starch glycolate, croscarmellose sodium, sodium carboxymethylcellulose and sodium alginate) using an 8 mm round punch and 2 ton compression force. The effects of the tablets on local pH were compared upon placement of the tablets in 0.01 M HCl solution (pH 2.0, 37° C.). pH was determined by adding an indicator to the solution.

The sodium starch glycolate tablet rapidly swelled and disintegrated, and produced a strong local acid-neutralizing effect; the croscarmellose sodium tablets also rapidly disintegrated, but produced a weaker local acid-neutralizing effect; the sodium carboxymethylcellulose tablets swelled slowly and exhibited some local acid-neutralization; and the sodium alginate tablets remained intact and exhibited little local acid-neutralization (data not shown).

In addition, a control tablet prepared with hydroxypropyl methylcellulose (HPMC), a polymer with no anionic groups, was tested in a similar manner, and exhibited no observable effect on pH.

Because of the gradual hydration of the tablets, the acid-neutralizing effect of the polymers in the swollen matrix may be inhomogeneous. The pH at the different locations of the tablet matrix was therefore assessed in additional sets of experiments, as follows.

200 mg tablets were prepared from sodium alginate, Na-CMC, CCS or sodium starch glycolate (SSG) with a 10 mm round punch and 1 ton compression force. Each tablet was placed at the center of a dish containing 100 ml 0.01 M HCl solutions (pH 2.0, room temperature), and after hydration, local pH was determined at four different points of the swollen tablet matrix, as shown in FIGS. 3A (for SSG), 3B (for CCS), 3C (for Na-CMC; CMC-Na) and 3D (for sodium alginate; Alg-Na), and as follows: (i) in the HCl solution outside of the formed gel boundaries (about 2-3 cm from the swollen matrix's surface); (ii) at the surface of the swollen matrix; (iii) at an intermediate distance between the surface and the center of the swollen matrix (about 0.5-1 cm from each of the surface and the center); and (iv) at the center of the swollen matrix). Measurement of the local pH inside the tablets was enables by using a pH meter with a thin electrode (as described under the “Materials and Methods” section).

As shown in FIGS. 3A-D, the SSG tablet rapidly swelled and disintegrated, and produced a strong local acid-neutralizing effect reaching pH 5.7 in the center of the swollen matrix (FIG. 3A). The CCS tablet also rapidly swelled, and produced a slightly weaker local acid-neutralizing effect (FIG. 3B). The CMC-Na tablet swelled slowly and exhibited strong local acid-neutralization (FIG. 3C); and the Alg-Na tablet exhibited the slowest swelling rate and local acid-neutralization similar to CCS (FIG. 3D). For all the tested polymers, the pH values measured in the center of the swollen matrix was higher than other areas, further supporting a concentration-dependent acid-neutralizing capacity of the polymers.

The results of these two sets of experiments show that for all the tested alkaline polymers, an increase in the local pH inside the matrix is achieved, up to pH of about 5-6. Such pH values are in line with the desired pH for maintaining SNAC in a dissolvable salt form, as the pKa of SNAC is about 5. This pH increase thus reduces precipitation of SNAC and consequently achieves higher concentrations of dissolved SNAC which are required for its action (and other absorption enhancers) as an absorption enhancer. The local pH increase also inhibits peptide degradation by pepsin (which is active only at low pH). These results further indicate that an alkaline (basic) polymer, by exhibiting limited diffusion in acidic solution and thus affecting pH primarily in a local manner, can result in a stronger and/or more prolonged effect on pH.

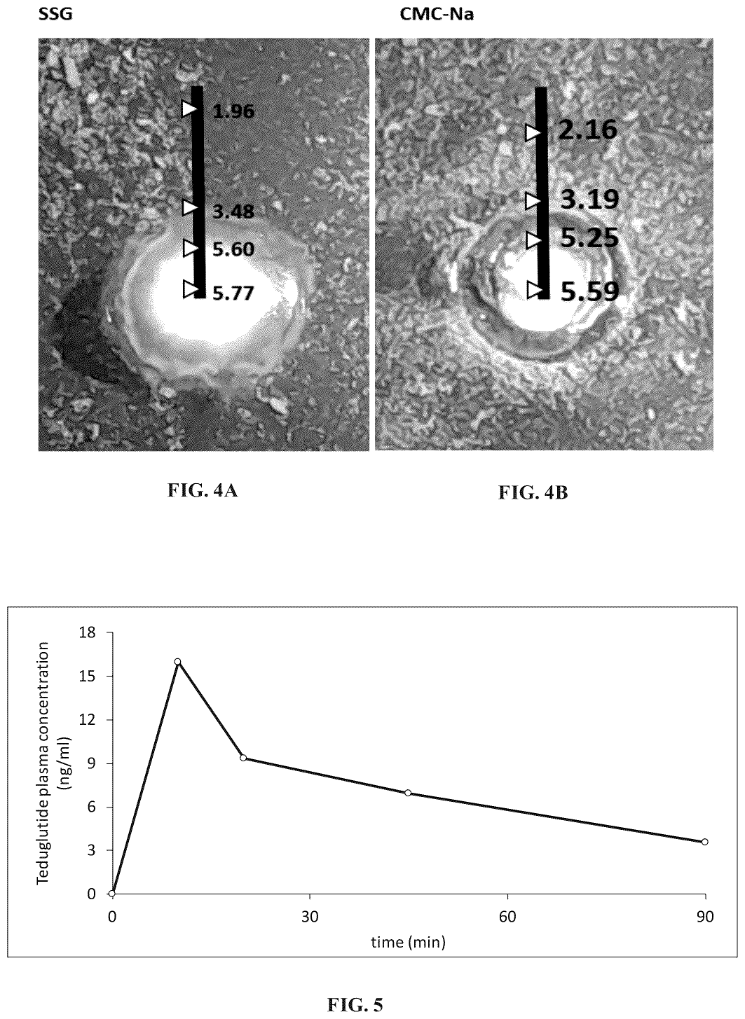

In yet another set of experiments, SSG, as an example of a fast swelling polymer that produces a high local pH, and CMC-Na, as an example of slow swelling polymer that produces a high local pH, were further tested in porcine gastric juice (withdrawn endoscopically from a sedated adult female domestic pig, sus scrofa domesticus). 200 mg tablets were prepared from each polymer with a 10 mm round punch and 1 ton compression force. Each tablet was placed at the center of a dish containing 100 ml of the porcine gastric juice at room temperature, and after hydration (wetting), local pH was determined at four different points of the swollen tablet matrix (i)-(iv) as explained above for the data shown in FIGS. 3A-D. The results are presented in FIG. 4A for SSG and in FIG. 4B for CMC-Na and are similar to those shown in FIGS. 3A and 3C, respectively.

Taken together, the above results indicate that alkaline group-containing polymers, especially sodium starch glycolate and croscarmellose sodium, can effectively neutralize acid within a local region (e.g., within the stomach), and thereby reduce protonation and inactivation of SNAC, within such a region. This phenomenon would be of particular importance when the polymer, SNAC and a peptide or polypeptide are co-localized (e.g., within a single dosage form), allowing prolonged SNAC-induced absorption of the peptide or the polypeptide.

Taken together with available data (not shown) that demonstrate that degradation of peptides by pepsin is inhibited at pH of 6 and higher, a dual local effect is achieved.

### Example 2

### Effect of Sodium Starch Glycolate on GLP-2 Absorption In Vivo Model

The ability of sodium starch glycolate (SSG) and/or croscarmellose sodium to facilitate drug absorption is assessed in an in vivo model (optionally a rat, dog and/or pig model), using teduglutide, apraglutide and/or glepaglutide as an exemplary form of GLP-2 and SNAC as model absorption enhancer.

Formulations are optionally in a form of tablets prepared by application of pressure to a powder mixture, optionally wherein the tablets have about the same amount of GLP-2 and SNAC, but differ in carboxylate-containing polymer being present (e.g., at a concentration of about 20 or about 30 weight percent) or absent.

Test animals are divided into groups which receive (optionally by intra-gastric gavage) different formulations e.g., comprising the GLP-2 and SNAC, with or without the carboxylate-containing polymer or with SNAC alone; and are optionally deprived of food the night before the experiments and during the experiment, and/or deprived of water an hour before the experiment and during the experiment. Blood is taken at predetermined time points, and levels of the tested GLP-2 (e.g., in plasma) are quantified by a suitable technique (optionally an ELISA method).

Teduglutide is a commercially available 33-amino acid peptide with significantly higher stability to metabolic degradation than the endogenous glucagon like peptide 2 (GLP-2). It is clinically used as a treatment for short bowel syndrome. Currently practiced methodologies employing teduglutide include subcutaneous (SC) injection, since its instability in and impermeability from the GI tract renders the drug's oral (PO) bioavailability negligible.

In an exemplary in vivo rat model, the effect of SNAC, as an exemplary absorption enhancer, and SSG, as an exemplary alkaline polymer, on the oral bioavailability of teduglutide was tested.

A formulation was prepared with 0.7 mg teduglutide (2.34 mg/Kg), 15 mg SNAC, 0.2 mg stearate and 5.33 mg SSG. All substances were in a dry powder form and mixed geometrically. Afterwards, mini-tablets were prepared from the mixes using a 2 mm punch and die set at 1 ton/cm².

The study was conducted in 7 male Sprague-Dawley (SD) rats weighing about 299 grams (290-320 grams). Animals were deprived of food 12-15 hours prior to administration. Water supply was removed one hour before administration and returned one hour post-administration. The mini-tablets were administered by a flexible intragastric gavage with a tip adapted for solid dosage forms administration, without the aid of water. Blood samples were thereafter collected at pre-determined time points, the plasma was separated, and the plasma level of teduglutide was quantified by an ELISA kit for the detection of endogenous GLP-2 in plasma (Abcam, Cat. No. ab222863) according to manufacturer's method Version 7, upon required modifications.

The obtained profile of teduglutide's plasma concentrations as a function of time is presented in FIG. 5, demonstrating that combination of SNAC with the buffering polymer (SSG) can be utilized to enable oral delivery of teduglutide.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A pharmaceutical composition comprising, as a therapeutically active agent, a glucagon-like peptide-2; and an absorption enhancer, and a polymer comprising a plurality of alkaline groups, wherein a concentration of said polymer in the composition is at least 10 weight percent of the total weight of the composition, said absorption enhancer being a substituted or non-substituted fatty acid or a salt thereof.

2. (canceled)

3. The composition of claim 1, wherein at least a portion of said alkaline groups are carboxylate groups.

4. The composition of claim 2, wherein at least a portion of said carboxylate groups are in a form of a pharmaceutically acceptable salt.

5-6. (canceled)

7. The composition of claim 1, wherein said absorption enhancer comprises NAC or a salt thereof.

8. The composition of claim 1, wherein a concentration of said absorption enhancer is at least 50 weight percent.

9. The composition of claim 1, wherein a concentration of said polymer is at least 20 weight percent.

10. The composition of claim 1, wherein a total concentration of said absorption enhancer and said polymer is at least 80 weight percent.

11. The composition of claim 1, wherein a concentration of said alkaline groups in the composition is at least 0.1 millimoles per gram.

12. The composition of claim 1, wherein said polymer is a crosslinked polymer.

13. The composition of claim 1, wherein said polymer comprises a polysaccharide.

14. The composition of claim 13, wherein said polysaccharide is selected from a starch derivative and a cellulose derivative.

15. The composition of claim 1, wherein said polymer comprises carboxymethyl groups.

16. The composition of claim 1, wherein said polymer is characterized by a pKa in a range of from 1.2 to 7.5.

17-18. (canceled)

19. The composition of claim 1, wherein said therapeutically active agent is selected from teduglutide, apraglutide, dapiglutide and glepaglutide.

20. The composition of claim 1, being in a form of a unit dosage form.

21. The composition of claim 20, wherein an amount of said alkaline groups in the unit dosage form is at least 0.03 millimoles.

22. (canceled)

23. The composition of claim 1, wherein said unit dosage form comprises one or more tablet.

24. A method of treating a medical condition in a subject in need thereof, the method comprising orally administering to a subject in need thereof the composition of claim 1, wherein said medical condition is treatable by said therapeutically active agent.

25. The method of claim 24, wherein said condition is selected from chemotherapy-induced adverse effects, cul-de-sac syndrome, digestion disorders, enteritis, inflammatory bowel disease, malabsorption syndromes, malnutrition, short-bowel syndrome, small intestine damage, celiac sprue, tropical sprue, hypogammaglobulinemic sprue, ulcers, and ulcerative colitis.

26. (canceled)
